(19) United States
(12) Patent Application Publication
(10) Pub. No.: US 2026/0137338 A1
(43) Pub. Date: May 21, 2026

(54) BIOMETRIC WEARABLE SYSTEMS AND METHODS FOR DETECTING INTENDED LIMB MOTION AND INVOKING PAIN MITIGATION THEREOF

(71) Applicant: COAPT LLC, Chicago, IL (US)

(72) Inventors: Blair Andrew Lock, Wilmette, IL (US); Levi John Hargrove, Chicago, IL (US)

(21) Appl. No.: 18/951,064

(22) Filed: Nov. 18, 2024

(51) Int. Cl. A61B 5/00

(52) U.S. Cl. CPC A61B 5/4824; A61B 5/6802

(57) ABSTRACT

Biometric metric wearable systems and methods are disclosed for detecting intended limb motion and invoking pain mitigation. Biometric signal data of a user is sensed by biosensor(s) that form part of respective sensor pod(s) of a biometric wearable device while the user performs an intended limb motion. The sensor pod(s) include biofeedback indicator(s). Processor(s) communicatively connected to the biometric wearable device analyze the biometric signal data to learn in real time or near real time user-specific biosignal pattern(s) detected when the user performs the intended limb motion, and generates therefrom learned user intent data configured for at least one of: (1) visualizing a virtual limb, (2) visually controlling a graphical aspect, and/or (3) visualizing the one or more user-specific biosignal patterns on a display device. The processor(s) then visualize the learned user intent data on the display device for invoking a pain mitigation effect with the user corresponding to the given limb.

### FIELD OF THE DISCLOSURE

The present disclosure generally relates to biometric wearable systems and methods, and more particularly to biometric wearable systems and methods for detecting intended limb motion and invoking pain mitigation for a give user. The pain mitigation effect may comprise reducing at least one ailment as experienced by the user comprising, by way of non-limiting example: limb pain, complex regional pain syndrome (CRPS), post-traumatic stress disorder (PTSD), or burning sensation(s).

### BACKGROUND

In the event that a user has either an amputated extremity, a bilateral amputation, a physical deformity, or a pathophysiology relating to the neurological or muscular control of extremities, traditional technologies lack adaptability to provide a user with user-specific pain mitigation treatment. Such users can suffer from a variety of ailments, and current techniques for addressing the ailments can be either non-user specific or difficult to administer in an efficient matter. For example, existing techniques for creating a visual reference for the purpose of treating ailments, such as “phantom limb pain” (PLP), have been ineffective. For example, traditional methods of treating PLP involve using a reference technology, such as a mirror or camera, to monitor both an injured and an uninjured extremity. In such traditional treatments, a user is instructed to move an uninjured extremity while simultaneously attempting to activate the muscles of an injured, typically amputated, extremity. The existing technology then recreates the image of how the injured extremity would have appeared to the user, superimposing the image over the amputated extremity as if the amputated extremity had not been amputated. This technique of recreating a visual image of an extremity that otherwise doesn't exist has demonstrated increasingly supported neurological value in reducing the amount of “phantom pain” that a user perceives for an amputated body part. However, the above technique is typically computationally expensive to perform and/or does not treat a user's specific aliment, which can be different from user to user.

For the foregoing reasons there is a need for biometric wearable systems and methods for detecting intended limb motion and invoking pain mitigation to provide for a specific user treatment.

### SUMMARY

The biometric wearable systems and methods as described herein provide detection of intended limb motion and innovation of pain mitigation customized for specific users having specific aliments. The biometric wearable systems and methods comprise a biometric wearable systems and methods that includes sensors for sensing user-specific biometric signal data. Such data may be transmitted to a cloud-based platform and stored with other user data.

Artificial intelligence (AI), such as machine learning, can be used to train an AI model for analyzing and output predictions and/or classification for detecting user-specific biosensor or otherwise biosignal patterns detected when the user performs the intended limb motion. Such analyzing may be used to generate learned user intent data for visualizing a virtual limb, visually controlling a graphical aspect (e.g., within a video game), and/or visualizing one or more user-specific biosensor patterns and/or otherwise biosignal patterns on a display device, each of which can be designed to treat limb pain. For example, the learned user intent data may be customized for a given user to invoke the pain mitigation response for the user to treat the user's specific limb or otherwise medical condition.

In addition, the biometric wearable systems and methods described herein can be configured to measure the user's intention to activate muscles corresponding to an amputated extremity, regardless of whether or not motion is produced by an intention to activate those muscles, which may be non-existent (e.g., due to amputation).

The present disclosure further describes biometric wearable systems and methods for treating medical conditions, where the biometric wearable systems and methods comprise capabilities to adapt to a user's unique condition, and by so doing, provide improvements over the prior art that lacked the ability to adapt to user-specific conditions or states. Each of these embodiments is further described herein.

In some aspects, the techniques described herein relate to a biometric wearable system configured to detect intended limb motion and invoke pain mitigation, the biometric wearable system including: a biometric wearable device including plurality of sensor pods, each sensor pod of the plurality of sensor pods including one or more biosensors configured to sense biometric signal data of a user, and further including one or more biofeedback indicators; one or more processors communicatively coupled to the one or more biosensors; a memory storing computing instructions that when executed by the one or more processors to cause the one or more processors to: sense the biometric signal data of the user from the one or more biosensors while the user performs an intended limb motion of a given limb of the user, analyze the biometric signal data to learn in real time or near real time a user-specific limb motion intent based on one or more user-specific biosignal patterns detected when the user performs the intended limb motion, generate, based on the user-specific limb motion intent, learned user intent data for at least one of: (1) visualizing a virtual limb on a display device, (2) visually controlling a graphical aspect on the display device, (3) visualizing the one or more user-specific biosignal patterns on the display device, and/or (4) visualizing the user-specific limb motion intent data on the display device, and visualize the learned user intent data on the display device for invoking a pain mitigation effect with the user corresponding to the given limb.

In some aspects, the techniques described herein relate to a biometric wearable system, wherein the pain mitigation effect reduces at least one ailment as experienced by the user including: (a) limb pain, (b) phantom limb pain, (c) complex regional pain syndrome (CRPS), (d) post-traumatic stress disorder (PTSD), or (e) burning sensation.

In some aspects, the techniques described herein relate to a biometric wearable system, wherein the display device is a screen device or a headset device.

In some aspects, the techniques described herein relate to a biometric wearable system further including: a transceiver communicatively coupled to the one or more processors, wherein the learned user intent data is transmitted via the transceiver to the display device.

In some aspects, the techniques described herein relate to a biometric wearable system, wherein the virtual limb or the one or more user-specific biosignal patterns and/or specific limb motion intent data is visualized in a virtual environment.

In some aspects, the techniques described herein relate to a biometric wearable system, wherein the display device is a display screen connected to the biometric wearable device, and wherein the virtual limb or one or more user-specific biosignal patterns and/or specific limb motion intent data is visualized on the display screen.

In some aspects, the techniques described herein relate to a biometric wearable system, wherein the computing instructions, when executed by the one or more processors further cause the one or more processors to implement at least one of: receive an input from a computing device to configure or alter an operation or setting of the biometric wearable device; and/or provide an indication of an operation or status of the biometric wearable device via at least one of the one or more biofeedback indicators of at least one of the plurality of sensor pods.

In some aspects, the techniques described herein relate to a biometric wearable system, wherein the plurality of sensor pods is part of a wearable prosthetic liner interface and/or a partial sleeve interface.

In some aspects, the techniques described herein relate to a biometric wearable system, wherein the one or more biofeedback indicators of each sensor pod are configured to provide an output to indicate amplitude information of a signal detected by the one or more biosensors of the sensor pods.

In some aspects, the techniques described herein relate to a biometric wearable system, wherein the output of the one or more biofeedback indicators of each sensor pod includes an illumination, in a predetermined pattern, to indicate amplitude information of a signal detected by the one or more biosensors of the sensor pods.

In some aspects, the techniques described herein relate to a biometric wearable system, wherein the intended limb motion includes a muscle bulge of the given limb of the user causing an increase of pressure of the given limb against the plurality of sensor pods, wherein the increased pressure invokes a sensorimotor effect that enhances the pain mitigation effect.

In some aspects, the techniques described herein relate to a biometric wearable system, wherein the increase in muscle bulge causes a corresponding increase in the user's biosignal output causing at least one of: (1) an illumination of a plurality of light emitting diodes of at least one sensor pod of the plurality of sensor pods, and/or (2) a frequency or vibration of actuators of at least one sensor pod of the plurality of sensor pods, wherein the increased pressure and the increase in the output invokes a sensorimotor effect that enhances the pain mitigation effect.

In some aspects, the techniques described herein relate to a biometric wearable system further including a transceiver, wherein data including any one or more of: (1) the biometric signal data of the user, (2) the one or more user-specific biosignal patterns and/or a user-specific limb motion intent of the user, and/or (3) the learned user intent data, and/or (4) data associated with the biometric signal data of the user, is configured to be transmitted to and from a server remote to the biometric wearable device.

In some aspects, the techniques described herein relate to a biometric wearable system, wherein the data provided to an artificial intelligence (AI) algorithm for training or retraining an AI model configured to analyze the biometric signal data to identify in real time or near real time the one or more user-specific biosignal patterns and/or a user-specific limb motion intent as detected when the user performs the intended limb motion.

In some aspects, the techniques described herein relate to a biometric wearable method for detecting intended limb motion and invoking pain mitigation, the biometric wearable method including: sensing, by one or more biosensors, biometric signal data of a user while the user performs an intended limb motion of a given limb of the user, wherein each of the one or more biosensors form part of one or more sensor pods of a biometric wearable device worn by the user, and wherein each the one or more sensor pods includes one or more biofeedback indicators; analyzing, by one or more processors, the biometric signal data to learn in real time or near real time a user-specific limb motion intent based on one or more user-specific biosignal patterns detected when the user performs the intended limb motion; generating, by the one or more processors based on the user-specific limb motion intent, learned user intent data for at least one of: (1) visualizing a virtual limb on a display device, (2) visually controlling a graphical aspect on the display device, (3) visualizing the one or more user-specific biosignal patterns on the display device, and/or (4) visualizing the user-specific limb motion intent data on the display device; and visualizing, by the one or more processors, the learned user intent data on the display device for invoking a pain mitigation effect with the user corresponding to the given limb.

In some aspects, the techniques described herein relate to a biometric wearable method, wherein the pain mitigation effect reduces at least one ailment as experienced by the user including: (a) limb pain, (b) phantom limb pain, (c) complex regional pain syndrome (CRPS), (d) post-traumatic stress disorder (PTSD), or (e) burning sensation.

In some aspects, the techniques described herein relate to a biometric wearable method, wherein the display device is a screen device or a headset device.

In some aspects, the techniques described herein relate to a biometric wearable method further including transmitting the learned user intent data via a transceiver to the display device.

In some aspects, the techniques described herein relate to a biometric wearable method, wherein the virtual limb or the one or more user-specific biosignal patterns and/or specific limb motion intent data is visualized in a virtual environment.

In some aspects, the techniques described herein relate to a biometric wearable method, wherein the display device is a display screen connected to the biometric wearable device, and wherein the virtual limb or one or more user-specific biosignal patterns and/or specific limb motion intent data is visualized on the display screen.

In some aspects, the techniques described herein relate to a biometric wearable method further including: receiving an input from a computing device to configure or alter an operation or setting of the biometric wearable device; and/or providing an indication of an operation or status of the biometric wearable device via at least one of the one or more biofeedback indicators of at least one of the one or more sensor pods.

In some aspects, the techniques described herein relate to a biometric wearable method, wherein the one or more sensor pods is part of a wearable prosthetic liner interface and/or a partial sleeve interface.

In some aspects, the techniques described herein relate to a biometric wearable method, wherein the one or more biofeedback indicators of each sensor pod are configured to provide an output to indicate amplitude information of a signal detected by the one or more biosensors of the sensor pods.

In some aspects, the techniques described herein relate to a biometric wearable method, wherein the output of the one or more biofeedback indicators of each sensor pod includes an illumination, in a predetermined pattern, to indicate amplitude information of a signal detected by the one or more biosensors of the sensor pods.

In some aspects, the techniques described herein relate to a biometric wearable method, wherein the intended limb motion includes a muscle bulge of the given limb of the user causing an increase of pressure of the given limb against the one or more sensor pods, wherein the increased pressure invokes a sensorimotor effect that enhances the pain mitigation effect.

In some aspects, the techniques described herein relate to a biometric wearable method, wherein the increase in muscle bulge causes a corresponding increase in the user's biosignal output causing at least one of: (1) an illumination of a plurality of light emitting diodes of at least one sensor pod of the plurality of sensor pods, and/or (2) a frequency or vibration of actuators of at least one sensor pod of the plurality of sensor pods, wherein the increased pressure and the increase in the output invokes a sensorimotor effect that enhances the pain mitigation effect.

In some aspects, the techniques described herein relate to a biometric wearable method further including transmitting via a transceiver, to and from a server remote to the biometric wearable device, data including any one or more of: (1) the biometric signal data of the user, (2) the one or more user-specific biosignal patterns and/or a user-specific limb motion intent of the user, and/or (3) the learned user intent data, and/or (4) data associated with the biometric signal data of the user, is configured to be transmitted to and from a server remote to the biometric wearable device.

In some aspects, the techniques described herein relate to a biometric wearable method further including providing the server to an artificial intelligence (AI) algorithm for training or retraining an AI model configured to analyze the biometric signal data to identify in real time or near real time the one or more user-specific biosignal patterns and/or a user-specific limb motion intent as detected when the user performs the intended limb motion.

In some aspects, the techniques described herein relate to a tangible, non-transitory computer-readable medium storing instructions for detecting intended limb motion and invoking pain mitigation, that when executed by one or more processors cause the one or more processors to: sense, by one or more biosensors, biometric signal data of a user while the user performs an intended limb motion of a given limb of the user, wherein each of the one or more biosensors form part of one or more sensor pods of a biometric wearable device worn by the user, and wherein each the one or more sensor pods includes one or more biofeedback indicators; analyze, by one or more processors, the biometric signal data to learn in real time or near real time a user-specific limb motion intent based on one or more user-specific biosignal patterns detected when the user performs the intended limb motion; generate, by the one or more processors based on the user-specific limb motion intent, learned user intent data for at least one of: (1) visualizing a virtual limb on a display device, (2) visually controlling a graphical aspect on the display device, (3) visualizing the one or more user-specific biosignal patterns on the display device, and/or (4) visualizing the user-specific limb motion intent data on the display device; and visualize, by the one or more processors, the learned user intent data on the display device for invoking a pain mitigation effect with the user corresponding to the given limb.

The present disclosure relates to improvement to other technologies or technical fields at least because the systems and methods disclosed herein allows a wearable biometric system to visualize learned user intent data based on an user intended motion to move one or more muscles at greater accuracy and efficiency than conventional techniques, especially where the systems and methods disclosed herein involve analyzing biometric signal data to learn in real time or near real time one or more user-specific biosignal patterns detected when the user performs an intended limb motion. Detection of such user-specific biosignal patterns may use a trained AI model to output predictions and/or classifications that indicate, with improved accuracy, the intended limb action of the user.

In addition, the present disclosure includes applying certain aspects or features, as described herein, with, or by the use of, a particular machine, e.g., a biometric wearable device to sense biometric signal data of a user from one or more biosensors while the user performs an intended limb motion of a given limb.

The present disclosure includes effecting a transformation or reduction of a particular article to a different state or thing, e.g., transformation or reduction of biometric signal data of a user into a medium easily used to generate, based on the one or more user-specific biosignal patterns, learned user intent data configured for at least one of: (1) visualizing a virtual limb on a display device, (2) visually controlling a graphical aspect on the display device, (3) visualizing the one or more user-specific biosignal patterns on the display device, and/or (4) controlling output of the display device based on the user-specific limb motion intent.

The present disclosure includes specific features other than what is well-understood, routine, conventional activity in the field, or adding unconventional steps that confine the claim to a particular useful application, e.g., biometric wearable systems and methods for detecting intended limb motion and invoke pain mitigation.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The figures described below depict various aspects of the system and methods disclosed therein. It should be understood that each Figure depicts an embodiment of a particular aspect of the disclosed system and methods, and that each of the Figures is intended to accord with a possible embodiment thereof. Further, whenever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and instrumentalities shown, wherein:

FIG. 1A illustrates an example biometric wearable system configured to detect intended limb motion and invoke pain mitigation, in accordance with various embodiments herein.

FIG. 1B illustrates a limb of a user wearing a biometric wearable device, in accordance with various embodiments herein.

FIG. 2A is a diagram illustrating a first set of biometric data and/or signals of a user that may be sensed by the example biometric wearable system of FIG. 1A, in accordance with various embodiments herein.

FIG. 2B is a diagram illustrating a second set of biometric data and/or signals of a user that may be collected by the example biometric wearable system of FIG. 1A, in accordance with various embodiments herein.

FIG. 2C is a diagram illustrating a third set of biometric data and/or signals of a user that may be collected by the example biometric wearable system of FIG. 1A, in accordance with various embodiments herein.

FIG. 3 is a block diagram illustrating a further example biometric wearable system configured to detect intended limb motion and invoke pain mitigation as described with FIG. 1, and also illustrating a cloud-based network for receiving and transmitting data comprising biometric data of one or more users, in accordance with various embodiments herein.

FIG. 4 is a block diagram illustrating an example biometric wearable method for detecting intended limb motion and invoking pain mitigation, in accordance with various embodiments herein.

FIG. 5 illustrates an example user interface as rendered on a display screen of a computing device in accordance with various embodiments disclosed herein.

### DETAILED DESCRIPTION

While the systems and methods disclosed herein is susceptible of being embodied in many different forms, it is shown in the drawings and will be described herein in detail specific exemplary embodiments thereof, with the understanding that the present disclosure is to be considered as an exemplification of the principles of the systems and methods disclosed herein and is not intended to limit the systems and methods disclosed herein to the specific embodiments illustrated. In this respect, before explaining at least one embodiment consistent with the present systems and methods disclosed herein in detail, it is to be understood that the systems and methods disclosed herein is not limited in its application to the details of construction and to the arrangements of components set forth above and below, illustrated in the drawings, or as described in the examples. Methods and apparatuses consistent with the systems and methods disclosed herein are capable of other embodiments and of being practiced and carried out in various ways. Also, it is to be understood that the phraseology and terminology employed herein, as well as the abstract included below, are for the purposes of description and should not be regarded as limiting.

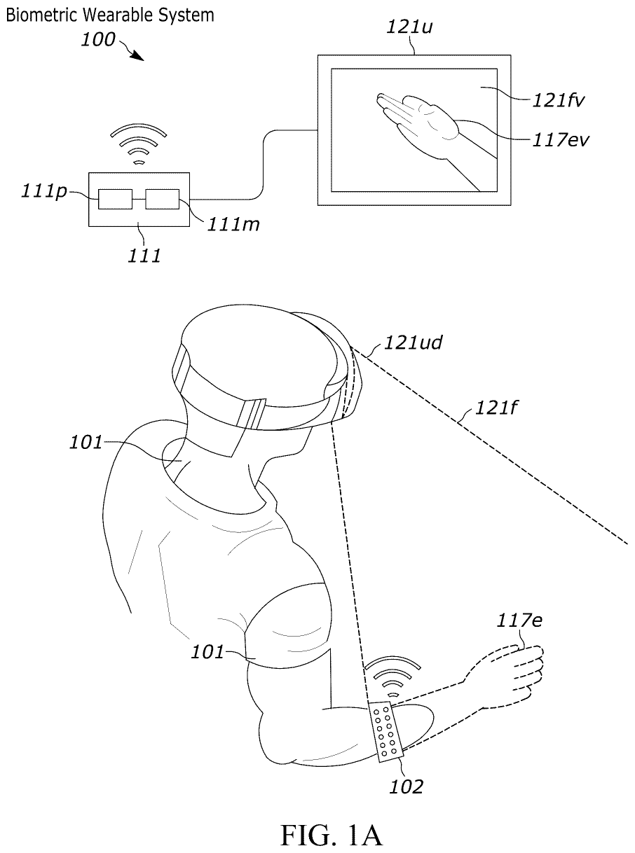

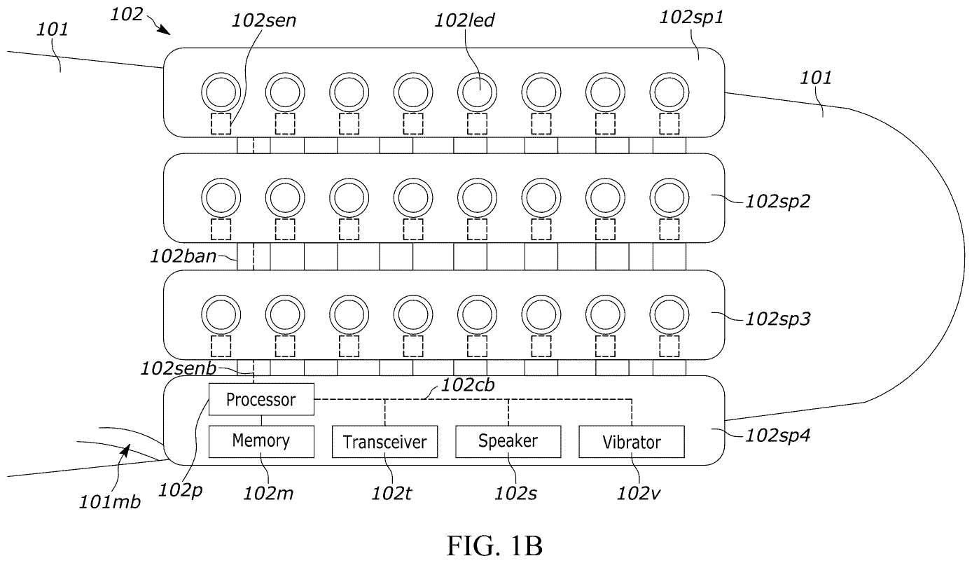

FIG. 1A illustrates an example biometric wearable system 100 configured to detect intended limb motion and invoke pain mitigation, in accordance with various embodiments herein. As shown for FIG. 1A, a biometric wearable system 100 is configured to sense biometric signal data of a user. FIG. 1A depicts a user 101 interfacing with biometric wearable system 100 while controlling a virtual limb (e.g., virtual limb 117ev) corresponding to a missing extremity 117e of the user 101, where the missing extremity 117e may be referred to herein as a phantom limb, and which may comprise an amputated limb, malformed limb, or otherwise non-whole extremity of the user 101. In the example of FIG. 1A, the user is viewing the virtual limb (e.g., virtual limb 117ev) via a user interface 121u of a user interface device 121ud. The biometric wearable system 100 comprises a biometric wearable device 102 configured to sense biometric signal data from the user's body. The user's field of view 121f is mapped to, or configured to correspond with, the user interface 121u to create or display a virtual field of view 121fv. The virtual field of view 121fv is displayed via the user interface 121 and contains, renders, or depicts the virtual limb (e.g., virtual limb 117ev), which is an amputated arm in the example of FIG. 1A. For example, virtual limb 117ev may be displayed on a display screen in 2D space (e.g., as rendered as 3D image in 2D space). Additionally, or alternatively, virtual limb 117ev may be displayed or rendered in 3D space, e.g., via a virtual reality (VR) headset. Additionally, the virtual limb 117ev may be depicted as a holographic image; appearing as a 2D or 3D image in the ordinary space as demonstrated by either a holographic projector or VR headset (e.g., a MICROSOFT MESH enabled device).

In the example of FIG. 1A, user interface device 121ud comprises a VR headset. The VR headset may comprise a VR headset such as META QUEST VR headset, an HP REVERB VR headset, a VALVE INDEX VR headset, a SONY PLAYSTATION VR headset, an HTC VIVE VR headset, a MICROSOFT MESH headset, or the like. In such embodiments, user interface 121u comprises a display screen of the VR headset as attached or included as part of the VR device (e.g., user interface device 121ud), where user interface 121u comprise a graphic user interface (GUI) capable of rendering VR graphics or images in virtual space via the user interface 121u of the VR headset.

Additionally, or alternatively, user interface device 121ud be or may further comprise a mobile device (e.g., a computing device 111), such as a cellular phone, tablet device, etc. such as an APPLE IPHONE device or GOOGLE ANDROID device. In such embodiments, the user interface 121u comprises a display screen of the mobile device as attached or included as part of the mobile device, where the user interface 121u comprises a graphic user interface (GUI) capable of rendering VR graphics or images on the display screen of the mobile device. For example, interface device 121ud may be an APPLE IPHONE device or GOOGLE ANDROID device having a display screen for rendering VR graphics or images on user interface 121u via, e.g., a GOOGLE CARDBOARD device and related app software as implemented on the mobile device, or the like.

In various embodiments, user interface device 121ud comprises, or is communicatively coupled to, one or more processors (e.g., a processor 111p), for executing computing instructions for rendering VR graphics or images, or for implementing any algorithms, methods, flowcharts, etc. as described herein. In addition, the interface device 121ud comprises, or is communicatively coupled to, one or more computer memories (e.g., a memory 111m), for storing instructions for rendering VR graphics or images, or for implementing any algorithms, methods, flowcharts, etc. as described herein. In various embodiments, the one or more computer memories 111m may comprise tangible, non-transitory computer-readable medium (e.g., RAM or ROM) for storing instructions, graphics, images, or the like.

In the embodiment of FIG. 1A, a computing device 111 comprises processor 111p communicatively coupled to memory 111m. In the depicted embodiment, processor 111p is communicatively coupled (via wireless signals) to biometric wearable device 102. Wireless signals may comprise any one or more of IEEE 802.11 wireless signals (WIFI), BLUETOOTH signals, or the like. Additionally, or alternatively, processor 111p may be communicatively coupled via wired signals, e.g., via a USB or similar wired connection (not shown) to biometric wearable device 102.

In various embodiments, biometric wearable system 100 includes software components that comprise computing instructions executable by a processor (e.g., processor 111p, 102p, and/or 304), and which may comprise computing instructions implemented in programming languages such as, e.g., C, C++, C #, GO, Java, Python, Ruby, R, or the like. The software component may be stored on a memory (e.g., memory 111m) communicatively coupled (e.g., via a system-on-a-chip (SoC) and/or computing bus architecture) to one or more processors (e.g., processor 111p, 102p, and/or 304). Processor 111p may be an ARM, ATOM, INTEL based processor, or other similar processor (e.g., as typically used with wearable or similar devices) for executing the computing instructions, applications, components, algorithms, source code, or otherwise software (e.g., of software component) as depicted or described herein for various methods.

Execution of the computing instructions of a software component by the processor 111p causes the processor 111p to perform an analysis of the biometric signal data (e.g., biometric signals and/or data 203, 210, and/or 210i) of the user 101 as sensed or otherwise detected by the biometric wearable device 102. For example, software component (stored in the memory 111m) may contain computing instructions executable by the processor 111p. The computing instructions may be compiled to execute on a processor (e.g., processor 111p, 102p, and/or 304) or may be otherwise be configured to be interpreted or run by the processor 111p. Such computing instructions may be coded to execute the algorithms, such as the methods and/or flowcharts as described herein. For example, computing instructions of a software component (e.g., stored in memory 111m) may comprise one or more event listeners, such as a listener function programmed to detect and/or receive biometric signal data of user e.g., biometric signals and/or data 203, 210, and/or 210i) as detected and/or received from the biometric wearable device 102. In this way, the biometric signal data of the user 101 would be pushed to, or otherwise received from, biometric wearable device 102 for sensing, detection, or generation of biometric signal data that would trigger the listener function to provide such biometric data for use for generation, based on user-specific limb motion intent, learned user intent data for at least one of: (1) visualizing a virtual limb on a display device, (2) visually controlling a graphical aspect on the display device, (3) visualizing the one or more user-specific biosignal patterns on the display device, and/or (4) controlling output of the display device based on the user-specific limb motion intent, or otherwise, as described herein.

It is to be understood that processor 111p and/or memory 111m may be differently configured, arranged, and/or coupled with respect to any of biometric wearable device 102, user interface device 121ud, and/or user interface 121u. For example, additionally, or alternatively, processor 111p and/or memory 111m may be incorporated into a medical device, such as prosthetic device, or other computing device communicatively coupled to biometric wearable device 102, user interface device 121ud, and/or user interface 121u, and configured to operate as part of biometric wearable system 100 and/or to implement biometric enabled virtual reality method(s) as described herein. For example, each of the biometric wearable device 102 (with its various sensors, as positioned with respect to the user), processor 111p, memory 111m, user interface device 121ud, user interface 121, etc. may be communicatively coupled to one another via a system-on-a-chip (SoC) architecture or other electronic architecture or interface, which may comprise a computing device (e.g., computing device 111) that includes hardware (e.g., processor 111p, 102p, and/or 304) of biometric wearable system 100 of and/or software (e.g., computing instructions as stored in memory 111m) for implementing the detection of intended limb motion and invoking of pain mitigation as described herein.

Additionally, or alternatively, biometric wearable device 102, processor 111p, memory 111m, and/or other user interface 121u may be part of separate computing devices, which are communicatively coupled, e.g., via a wired or wireless connection. For example, in one embodiment, user interface 121u may be implemented on a separate or remote computing device (e.g., a laptop or computer) in wireless communication (e.g., BLUETOOTH protocol or WIFI (802.11) standard) with the biometric wearable system 100, where a user configures the biometric wearable system 100 (e.g., by training or otherwise configuring the biometric wearable system 100, user interface 121u, or biometric wearable device 102 components and configuration as described herein) via the remote user interface 121u on the separate computing device. A biometric enabled virtual reality apparatus manager, comprising computing instructions, etc., may also be implemented or configured on separate computing device, to implement or control the biometric wearable systems and methods described herein.

FIG. 1B illustrates a limb (e.g., an arm) of a user 101 wearing a biometric wearable device 102, in accordance with various embodiments herein. The biometric wearable device 102 may comprise or otherwise be part of a biometric wearable system configured to detect intended limb motion and invoke pain mitigation as described and depicted, for example, for the biometric wearable system 100 in FIG. 1A.

In the example of FIG. 1B, biometric wearable device 102 comprises a plurality of sensor pods 102sp1, 102sp2, 102sp3, and 102sp4. The sensor pods 102sp1-102sp4 are secured via one or more bands (e.g., band 102ban), which can be flexible or rigid. Further, sensor pods 102sp1-102sp4 can be arranged or otherwise positioned to securely fit to user 101's limb (e.g., an arm with an amputation or otherwise arm with having a phantom limb status). In some aspects, the biometric wearable device 102 can be adjusted to firmly and/or securely fit to the user's limb to create a customized user fit biometric wearable device. It should be understood, however, that different and/or additional attachments or mechanisms may be used to attach the sensor pods 102sp1-102sp4 to one another and/or secure the biometric wearable device 102 to the user. For example, in some aspects, the plurality of sensor pods (e.g., sensor pods 102sp1-102sp4) may be part of a wearable prosthetic liner interface and/or a partial sleeve interface. In such aspects, the sensor pods maybe embedded, at least partially, within a prosthetic liner that can be fit, such as custom fit, to the user to allow for contact of the sensor pods with the user's body when the prosthetic is fit to the user's limb (e.g., an arm).

As shown for FIG. 1B, each sensor pod (e.g., each of sensor pods 102sp1 102sp4) includes one or more biosensors (e.g., biosensor 102sen) configured to sense biometric signal data of a user, e.g., when the user performs an intended limb motion of a given limb of the user. Each biosensor (e.g., biosensor 102sen) may comprise one or more of electromyographic electrode(s), infrared sensor(s), pressure sensor(s), electroencephalogram electrodes, electrooculogram sensor(s), and/or scleral search coil(s). Additionally, or alternatively, other sensor(s) may comprise force sensitive resistors (e.g., force myography), near infra-red spectroscopy (NIRS), mechanomyography (MMG), and sonomyography sensor(s). More generally, a sensor modality that measures a muscle contraction intention may be used. The biosensor(s) may be positioned at or near the user's skin for sensing biometric data of the user, for example, when the user 101 moves or otherwise activities his or her limb or muscle(s) thereof.

The one or more biosensors (e.g., biosensor 102sen) may be positioned at one or more location(s) relative to user 101 for sensing or otherwise collection of biometric signal data of the user. The positioning may comprise in proximity to user 101, in superficial contact with user 101, subcutaneously positioned to user 101, and/or subdermally implanted within the user 101, in accordance with various embodiments herein.

Certain advantages as to data fidelity, allowing for visualizing a virtual limb, visually controlling a graphical aspect, visualizing the one or more user-specific biosignal patterns on a display device, and otherwise are achieved through various sensor placements and locations, as illustrated by FIG. 1B. For example, the location(s) of the biometric sensors allow biometric wearable device 102 to collect biometric signal data of a user at different and/or various intensities or types, and/or at different and/or various fidelities to provide increased accuracy and/or different qualities of biometric signal data. Such increased accuracy and/or different qualities of biometric signal data provide the biometric wearable systems and methods described herein with exact or specific (e.g., user-specific) biometric signals or data in order to allow for precise and/or user-specific visualizing a virtual limb, visually controlling a graphical aspect, visualizing the one or more user-specific biosignal patterns on a display device, or otherwise as described herein.

Further, as shown for FIG. 1B, biometric wearable device 102 may comprise one or more processors communicatively coupled to the one or more biosensors. The one or more processors may communicate with one another to process biometric signal data of the user. In some aspects, each sensor pod may comprise a respective processor. In other aspects, only a subset of the sensor pods includes a respective processor. Still further, in alternative aspects, a single sensor pod may comprise a single processor, which is responsible for controlling the biometric wearable device 102 and receiving data from the user.

As shown in the example of FIG. 1B, sensor pod 102sp4 comprises processor 102p. Further, as shown, processor 102b is communicatively coupled to the one or more biosensors (e.g., biosensor 102sen) via a sensor bus (e.g., sensor bus 102senb), where processor 102p electronically receives sensor the biometric signal data of the user across sensor bus 102senb. In various aspects, the sensor bus may be included in one or more of the bands (e.g., band 102ban), where data from the various biosensor(s) may be computed to processor 102b, or other processors(s) of biometric wearable device 102 and/or biometric wearable system 100.

In various aspects, each of the sensor pods may electronically transmit the biometric signal data of the user to a processor (e.g., processor 102p) onboard the biometric wearable device 102. In some aspects, for example, the one or more(s) (e.g., band 102ban) may include respective sensor buses (e.g., sensor bus 102senb) for electronically delivering biometric signal data of user 101, e.g., in particular biometric signal data of the user 101's limb when the user moves or otherwise his or her limb or other body portion.

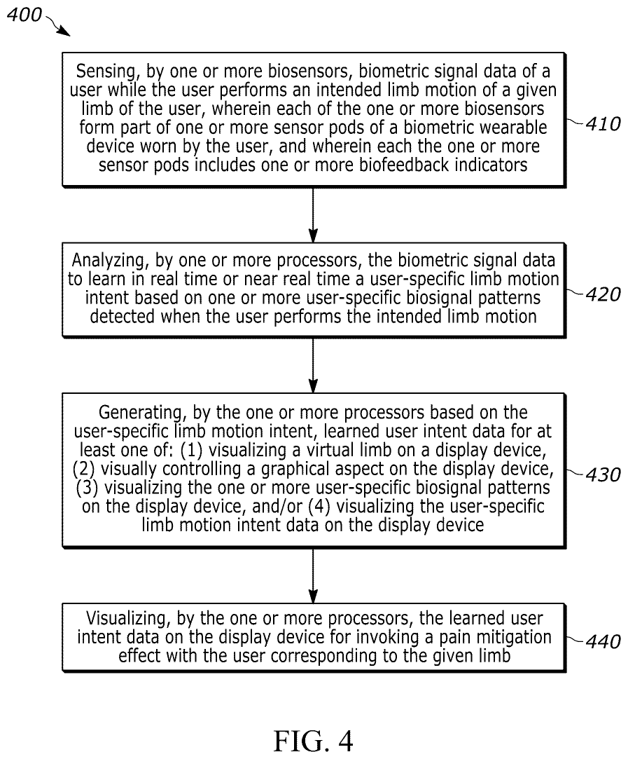

The one or more processors of a biometric wearable device (e.g., biometric wearable device) may be communicatively coupled to one or more memories. In one example, and as shown for FIG. 1B, processor 102p is communicatively couped to memory 102m. Memory 102m may store computing instructions that when executed by the processor 102p cause to implement or execute the algorithms, methods, or other functions as described herein, including, but not limited to, method 400 as described for FIG. 4.

Memory 102m may also store other data as described herein, for example biometric signal data of the user or data generated from such data. Still further, memory 102m may also store artificial intelligence (AI) models (e.g., AI model 308a) as described herein for receiving biometric signal data of user 101 as input and providing as output classifications or predictions user-specific biosignal patterns as performed by a specific user (e.g., user 101), where such output can be indicative of intended limb motions of the user 101.

Still further, in the example of FIG. 1B, each of the processors can be communicatively coupled to onboard devices or components. For example, processor 102b can be communicatively connected to transceiver 102t, speaker 102s, and vibrator 102v via a computer or otherwise electronic bus 102cb. Transceiver 102t may communicate with processor 102b to send and receive wireless signals, such as BLUETOOTH based signals, to other devices, including for example, user interface device 121ud, processor(s) 111p, user interface 121u, and/or other components, devices, servers, or systems as described herein. For example, in various aspects, transceiver 102t may be communicatively coupled to the one or more processors (e.g., processor 102p) and the one or more processors may transmit learned user intent data transmitted via transceiver 102t to a display device (e.g., user interface device 121ud). In some aspects the transceiver may receive input. For example, in some aspects, processor 102p may implement computing instruction to cause transceiver 102t to receive an input from a computing device (e.g., a computing device having processor(s) 111p) to configure or alter an operation or setting of the biometric wearable device 102.

Speaker 102s may provide audible feedback to the user and/or vibrator 102v may provide tactile feedback to a user when biometric signal data of a user is received or sensed, when a user-specific biosignal pattern is predicted or classified (e.g., by an AI model), and/or when it is detected that the user performs the intended limb motion, e.g., as described herein.

Further with reference to FIG. 1B, the biometric wearable device 102 may include one or more biofeedback indicators. In various aspects, processor 102p may provide an indication of an operation or status of the biometric wearable device 102 via at least one of the one or more biofeedback indicators of at least one of the plurality of sensor pods. For example, as shown for FIG. 1B, each sensor pod (e.g., each of sensor pods 102sp1-102sp4) includes a series of light emitting diodes (LEDs), such as LED 102led. The feedback indicators can provide feedback in visual fashion (e.g., illumination of the LEDS). Still further, each of the LEDs (e.g., LED 102led) can vary its visual indication (e.g., via illumination intensity, color, etc.) based on the biometric signal data sensed and received for the user. In this way, the biofeedback indicators can indicate where biometric data is received with respect to various positions on the user's limb (or other portion the user's body). Similarly, the biofeedback indicators can indicate a signal strength or otherwise amplitude information of the biometric data hat is sensed or received with respect to various positions on the user's limb.

For example, in various aspects the one or more biofeedback indicators (e.g., LED 102led) of each sensor pod (e.g., one or more of sensor pods 102sp1-102sp4) are configured to provide an output to indicate a strength or otherwise amplitude of a signal detected by the one or more biosensors of the sensor pods. In some aspects, the one or more biofeedback indicators may comprise audible outputs (e.g., such as one or beeps or tones), or tactile outputs (e.g., vibrations). More generally, the biofeedback indicators may comprise LEDs, LCD display(s), vibrotactile elements, speakers, buzzers, or the like.

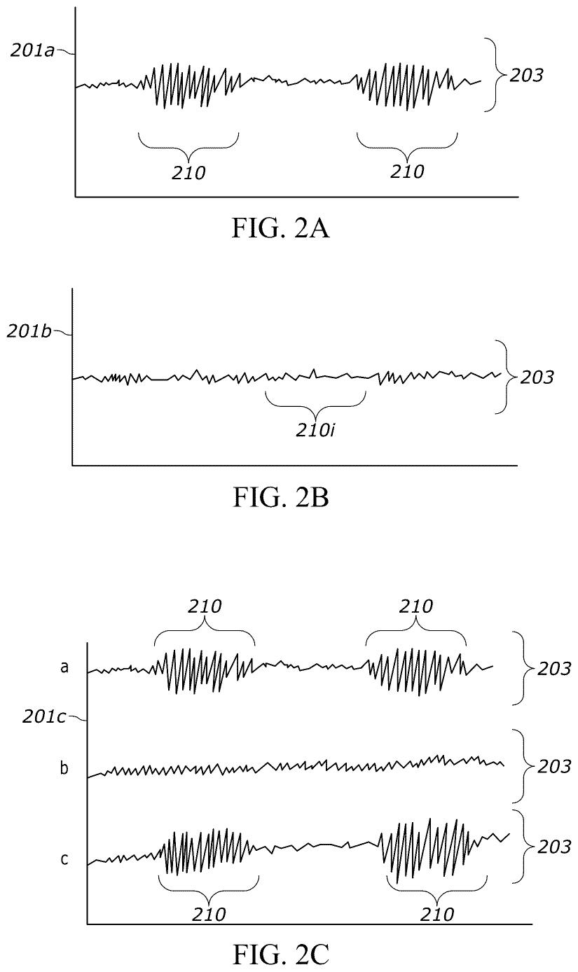

In still further aspects, output of the one or more biofeedback indicators of each sensor pod may comprise an illumination, in a predetermined pattern, to indicate a strength of a signal, amplitude (e.g., amplitude information such as peaks and troughs of biometric signal data as shown for FIGS. 2A-2C), and/or increase in muscle bulge detected by the one or more biosensors of the sensor pods. The illumination may be in one or more colors. In one example, the pattern may comprise turning on or otherwise enabling or providing electrical power to more of the biofeedback indicator(s) when the signal strength or otherwise amplitude information is high, and turning on fewer or otherwise enabling fewer of the biofeedback indicator(s) when the signal strength, amplitude information, and/or muscle bulge is low. Other patterns are also contemplated herein, including different colors and/or patterns across the one or more biofeedback indicators and/or sensor pods. For example, a different color may be illuminated for high signal strength, amplitude information, and/or muscle bulge compared to low signal strength, amplitude information, and/or muscle bulge. In other embodiments, certain biofeedback indicator(s) may be displayed on different sensor pods in order to form symbols or patterns, e.g., where the LEDs form a pattern indicating low signal strength, amplitude information, and/or muscle bulge in a given shape (e.g., a downward arrow, minus sign, or other such shape).

In some aspects, an intended limb motion may comprise a muscle bulge (e.g., muscle bulge 101mb) of the given limb of the user. The muscle bulge (e.g., muscle bulge 101mb) may cause an increase of pressure of a given limb against the plurality of sensor pods (e.g., one or more of sensor pods 102sp1-102sp4) and/or bands and/or other portion. In some aspects, such activity may cause an increase in signal strength or otherwise amplitude information detected by the one or more biosensors of the sensor pods. In such aspects, the increased pressure can invoke a sensorimotor effect for or of the user that enhances a pain mitigation effect. In this way, biometric wearable device 102 may be configured to sense, invoke, or otherwise cause of experience a muscle bulge (e.g., muscle bulge 101mb). Such configuration may comprise a positioning of the biometric wearable device 102 with respect to, or specifically in contact with, a particular muscle or muscle group of the user. The positioning of the biometric wearable device 102 may be accomplished by adjusting length(s), location(s), and/or elasticitie(s) of the one or more bands (e.g., band 102ban) and/or one or more of sensor pods 102sp1-102sp4.

In some aspects, the increase in signal strength or otherwise amplitude information, e.g., as caused by the muscle bulge (e.g., muscle bulge 101mb), can cause a corresponding increase in the output of biometric wearable device 102. For example, such output may comprise an illumination of a plurality of light emitting diodes of at least one sensor pod of the plurality of sensor pods. Additionally, or alternatively, as a further example, the output of biometric wearable device 102 may comprise a frequency or vibration of actuators of at least one sensor pod of the plurality of sensor pods (e.g., one or more of sensor pods 102sp1 102sp4). That is, for example, a vibrotactile actuator may comprise actuators that change vibration intensity and/or frequency in proportion to the biosensor data received from one or more of sensor pods 102sp1-102sp4. In each of these examples, increased pressure and the increase in the output can invoke a sensorimotor effect that enhances the pain mitigation effect for the user. That is, the sensorimotor stimuli added by the pressure of the biometric wearable device 102 (e.g., the sensor band(s) and/or sensor pods 102sp1-102sp4), the activated LEDs, and/or the vibrators can each alone, or together as a group, contribute to reduced pain for the user. In this way, there is a cause and effect of the user generating commands via his or her limb motion and receiving output (e.g., LED(s) lighting and/or vibrator(s) vibrating), where the increased pressure caused by the muscle bulge of pushing on the biometric wearable device 102 together with such output contribute to the pain mitigation effect attributable to the biometric wearable device 102.

FIGS. 2A-2C illustrate diagrams (e.g., diagrams 201a, 201b, and 201c) of sets of biometric signal data and/or signals of a user (e.g., user 101) that may be collected by the example biometric wearable system 102 as shown for FIGS. 1A and 1B. In particular, FIG. 2A is a diagram 201a illustrating a first set of biometric signal data and/or signals of a user (e.g., user 101) that may be sensed or collected by the example biometric wearable system of FIG. 1A, in accordance with various embodiments herein. Similarly, FIG. 2B is a diagram 201b illustrating a second set of biometric signal data and/or signals of a user (e.g., user 101) that may be collected by the example biometric wearable system of FIG. 1A, in accordance with various embodiments herein. In addition, FIG. 2C is a diagram 201c illustrating a third set of biometric signal data and/or signals of a user (e.g., user 101) that may be collected by the example biometric wearable system of FIG. 1A, in accordance with various embodiments herein.

Biometric wearable device 102, with its various sensors (e.g., positioned relative to user 101 as described herein for FIGS. 1A and/or 1B) may collect biometric signal data and/or signals of user 101. For example, as shown in FIG. 1A, biometric signal data and/or signals 203 are collected from a limb, such as an injured body component (e.g., amputated arm) of user 101. It is to be understood, however, that biometric signal data and/or signals may be collected from of a user at additional, or different, body components (e.g., either injured or non-injured), for example, at a user's leg, ankle, foot, arm, neck, head, and/or other body part and/or extremity. More generally, FIGS. 2A-2C demonstrate non-limiting different types of biometric signal data and/or signals that may be collected from a user (e.g., user 101), as used to create a user-specific profile (e.g., a physiological profile of a user) in relation to detected intentions of the user upon intending to activate one or more muscle groups of the user.

FIGS. 2A-2C further illustrate examples of biometric signals and/or data 203, 210, and/or 210i as detected for a user (e.g., user 101) and the user's intention to activate (or not activate) one or more muscles. In particular, FIGS. 2A-2C demonstrate examples where a biometric detection device (e.g., biometric wearable device 102) and/or its sensor(s) are in contact with user 101, for example, as shown for FIG. 1A and/or FIG. 1B. As shown for each of diagrams 201a-201c of FIGS. 2A-2C, biometric signals 203 of the user are received from the biometric wearable device 102. Such biometric signals 203 may be filtered or processed to become biometric signal data. As described herein, biometric signals and/or biometric signal data 203, 210, and/or 210i may be referred to interchangeably herein as biometric signal data, biometric signals, biometric data, biometric filtered signals, biometric filtered data, and the like. Such biometric signals may be analyzed by a processor (e.g., processor 111p, 102p, and/or 304) for the presence of one or more user intentions to activate corresponding one or more muscles. For example, diagrams 201a and 201c each illustrate biometric signal portions 210 that may be determined by a processor as indicating the presence of one or more user intentions to activate corresponding one or more muscles. The biometric signal portions 210 may represent an increased or intensified signal and/or data activity (as compared to a baseline non-activity) of the user's detected biometric signal data corresponding to the one or more user intentions to activate the user's one or more muscles. For example, the presence of the intention(s) to activate one or more muscles is created by the user 101 attempting to perform a motion (e.g., which can be in response to a user motion prompt), while in biometric contact with the biometric wearable device 102.

FIG. 2B (diagram 201b) demonstrates a series or set of idle biometric signals and/or data 210i of a user (e.g., user 101). Such signals may be received when the user is at rest. Such signals may be useful in generating or performing a baseline of the user (e.g., non-activity of a user). The baseline may be used as part of the user's physiological profile, which is user-specific to the user. For example, the baseline may comprise the absence of detection of, or low signal strength or otherwise amplitude information of, biometric signal data of the user across one or more particular sensors. Together, biometric signals and/or data 203, 210, and/or 210i may be used to define or otherwise represent a user-specific intention to activate one or more muscles and may be used in the analysis to determine whether or not the intention to activate one or more muscles is present.

FIG. 2C (diagram 201c) demonstrates multiple series or sets (set a, set b, and set c) of biometric signals and/or data (e.g., biometric signals and/or data 203, 210, and/or 210i) of a user (e.g., user 101). Each of the sets a, b, and c may be generated by different sensors of biometric wearable device 102, such as sensors as differently positioned as described herein. In some embodiments, the biometric signals and/or data 203, 210, and/or 210i may be combined (e.g., averaged, summed, etc.) to provide an overall signal of the user for visualizing a virtual limb, visually controlling a graphical aspect, visualizing the one or more user-specific biosignal patterns on a display device, or otherwise as described herein. Additionally, or alternatively, the biometric signals and/or data 203, 210, and/or 210i may be separately processed (e.g., by a processor) for visualizing a virtual limb, visually controlling a graphical aspect, visualizing the one or more user-specific biosignal patterns on a display device, or otherwise as described herein.

It is to be understood that biometric signals and/or data 203, 210, and/or 210i may be of analogue and/or digital form, where a processor (e.g., processor 111p, 102p, and/304) may be configured to analyze one or both analogue and/or digital signals. For example, in various embodiments processor (e.g., processor 111p, 102p, and/304) may be configured to receive analogue or raw signal data of a user as detected by biometric wearable device 102. A processor (e.g., processor 111p, 102p, and/304) may be configured to receive biometric signal data in digital form as processed or pre-processed by biometric wearable device 102. Still further, additionally, or alternatively, processor (e.g., processor 111p, 102p, and/304) may receive analogue or raw signal data of a user as detected by biometric wearable device 102 and process or filter such data to create digital data for additional use, analysis, determination, or as otherwise described herein.

Biometric signals and/or data 203, 210, and/or 210i, as described for FIGS. 2A-2C, illustrate the diversity of data collected from biometric wearable device 102. In various embodiments, biometric signals and/or data 203 may comprise electromyographic data (EMG). FIGS. 2A-2C demonstrate the biometric signals collected from a variety of, but non-limiting, biometric wearable device 102 configurations. In FIGS. 2A and 2C, the biometric signals which may be stored in a computer memory (e.g., computer memory 111m, 102m, and/or 306) as biometric signal data, and can define the user's intention to activate one or more muscles (e.g., as determined or detected from biometric signals and/or data 210). In each of FIGS. 2A-2C, the biometric signals and/or data (e.g., biometric signals and/or data 203, 210, and/or 210i, whether filtered or non-filtered) are recorded over time (x-axis) and may be compared against the amplitude, frequency, and/or magnitude of biometric signals detected by the biometric wearable device 102 (y-axis). Diagram 2C demonstrates two separate intentions (intentions a and c) to activate one or more muscles (e.g., as determined or detected from biometric signals and/or data 210) between moments of idle behavior (behavior b) on behalf of the user (e.g., user 101) to activate one or more muscles that correspond with the biometric detectors detecting biometric signals. FIG. 2B demonstrates biometric signal data 203 collected from a user (e.g., user 101) that is currently collecting idle biometric signals. Based on the contents of the signals collected from FIG. 2B, the processor (e.g., processor 111p, 102p, and/304) may determine after an analysis of muscle intention (e.g., analysis of muscle intentions 112 as described for FIG. 4 herein) that the user 101 has not intended to activate one or more muscles (e.g., as determined or detected from biometric signals and/or data 210i). The biometric signal data 203 as illustrated in FIGS. 2A-2C, demonstrates a non-limiting and specific configuration of biometric sensors detected by the biometric wearable device 102 at a given time period.

Additionally, or alternatively, FIGS. 2A-2C may each represent separate channels, which may each being individual biometric sensors (channels of data) that independently provide biometric signals from different locations around the user 101. While the user (e.g., user 101) initiates an intention to activate one or more muscles (e.g., as determined or detected from biometric signals and/or data 210), the biometric wearable device 102 may temporally record the biometric signals from or of the user (e.g., user 101) from across leads or sensors, for example as described herein for FIGS. 1B and/or 1C. Based on the biometric signal data 203, 210, and/or 210i recorded, the biometric signals from leads or sensors may be used to determine intentions to activate one or more muscles (e.g., as determined or detected from biometric signals and/or data 210 by processor (e.g., processor 111p, 102p, and/304)), respectively. In some instances, the biometric sensor recording data from a lead or sensor (e.g., as illustrated for FIG. 2B) may not pick up any intentions to activate one or more muscles (e.g., as would be determined or detected from biometric signals and/or data 210i), and the associated muscles would be considered to be idle as described herein.

Furthermore, FIGS. 2A-2C demonstrate a range of measurement fidelity of biometric wearable device 102 with respect to biometric signals of user 101. Such measurement fidelity may include multiple different locations about the user (e.g., as described for FIGS. 1A and 1CB, which may or not be temporally in tandem. Leads or sensors (e.g., as described for FIGS. 1B and 1C) may detect signals indicating the intention to activate one or more muscles (e.g., as determined or detected from biometric signals and/or data 210) in a similar temporal fashion. Based on the configuration of the biometric wearable system 100, e.g., which may be determined by the user 101 a user interface as described, processor (e.g., processor 111p, 102p, and/304) may determine that the intentions to activate one or more muscles (e.g., as determined or detected from biometric signals and/or data 210) correlate with a specific user muscle contract, motion, or intention. This information may then be used by the processor (e.g., processor 111p, 102p, and/304) to determine the extent to which the virtual limb 117ev is to visualize a virtual limb, visually controlling a graphical aspect, visualize the one or more user-specific biosignal patterns on a display device, and/or otherwise as described herein. In comparison, the biometric signal data in FIG. 2B illustrates the user (e.g., user 101) being idle, or not otherwise generating biometric signals to an extent that would indicate the intention to not activate one or more muscles (e.g., as determined or detected from biometric signals and/or data 210i).

Conversely, FIG. 2C may demonstrate the intention to activate one or more muscles (e.g., as determined or detected from biometric signals and/or data 210), whether or not the user (e.g., user 101) is able to actively move the muscles they are intending to activate. Because the biometric signals generated by the user (e.g., user 101), and as detected by the biometric wearable device 102, are not necessarily determinate that a motion is taking place, the user (e.g., user 101) may be intending to activate one or more muscles (e.g., as determined or detected from biometric signals and/or data 210) but unable to create the motion they are intending to conduct. A processor (e.g., processor 111p, 102p, and/304) may determine that the intention to activate one or more muscles (e.g., as determined or detected from biometric signals and/or data 210) is indicated through the biometric signal data 203 and/or 210 that is received by the biometric device 102, and initiate the process to create or determine a virtual representation of an intended motion irrespective of whether or not the user (e.g., user 101) was able to create the intended motion.

Still further, FIGS. 2A-2C illustrate biometric signal data as received (over time and recorded as signal data in a computer memory, temporally, or in segments), identified, or as otherwise detected when a user (e.g., user 101) is active, e.g., performing a gesture, motion, or intention to activate a muscle or a group of muscles to perform an action, such as a user motion prompt. Biometric signal data is detected over time via the biometric wearable device 102 temporally, as described herein, at various biometric signal strengths or otherwise amplitude information, which is as a whole, indicates a data pattern that defines or represents a user-specific or user selected motion, including, e.g., in response to a user motion prompt. In various embodiments, such data pattern may be used to generate, record, create, provide, or otherwise implement a given user-specific configuration, as controlled biometric wearable device 102, in order to configure the biometric wearable device 102, initial profile, or user interface device 121ud for specific use by the user. In some embodiments, such detection of biometric signal data may cause processor (e.g., processor 111p, 102p, and/304) to initiate visualizing a virtual limb, visually control a graphical aspect, visualizing the one or more user-specific biosignal patterns on a display device, or otherwise, including, for example, creating alternate visual cue to help the user 101 with the proprioception of graphics or visualization about the user's 101 current space, as depicted in the virtual visual field 121fv of the user in either real, holographic, 2D, or 3D virtual space.

In the examples of FIGS. 2A-2C, in some embodiments, a motion intention of the user (e.g., user 101), e.g., flexing the arm at the elbow, may create biometric signals that are consistent with recorded biometric signal data (e.g., as previously recorded in a memory, e.g., computer memory 111m, 102m, and/or 306). Such motion intention may be conducted on behalf of the user that is separate from another motion intention. For example, the flexion at the elbow motion may create biometric signals of the user, which may create a muscle bulge. As a whole and in a sequence, these user-specific and user-selected motions may be used by the processor (e.g., processor 111p, 102p, and/304) to generate or determine virtual representation of the intended motion. In some embodiments, the virtual representation will move virtual limb 117ev in real space or virtual space, e.g., as visible via user-interface 121u and/or user-interface 121ud.

Additionally, or alternatively, a user-specific motion may be defined as one or more unique motions or motion intentions as defined by the user, e.g., by configuration via the user-interface 121u. In some cases, the motion or motion intentions are defined by the user (e.g., user 101) or, in some embodiments, by a second user that has the capacity and/or authorization to input information into the biometric wearable system 100, to be stored in a memory (e.g., computer memory 111m, 102m, and/or 306). This allows a caregiver, provider, physician, or otherwise authorized person(s) to monitor, create, or define a profile for the user comprising biometric signal data as generated by the user (e.g., user 101).

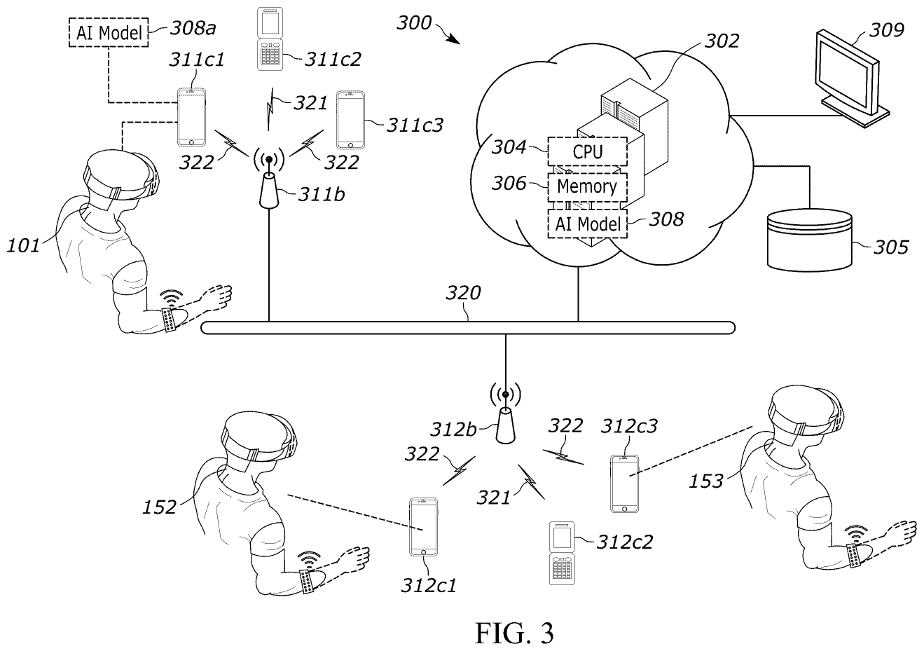

FIG. 3 is a block diagram illustrating a further example biometric wearable system configured to detect intended limb motion and invoke pain mitigation as described with FIG. 1, and also illustrating a cloud-based network 300 for receiving and transmitting data comprising biometric data of one or more users, in accordance with various embodiments herein. The cloud-based network 300 allows for transmission and receipt of data (e.g., biometric signal data, settings, gameplay scores, usage, etc.) from a given processor or device (e.g., processor(s) 111p and/or biometric wearable device 102) over a computer network to a cloud-based server.

In the example embodiment of FIG. 3, cloud-based network 300 includes server(s) 302, which may comprise one or more computer servers. In various embodiments server(s) 302 comprise multiple servers, which may comprise multiple, redundant, or replicated servers as part of a server farm. In still further embodiments, server(s) 302 may be implemented as cloud-based servers, such as a cloud-based computing platform. For example, server(s) 302 may be any one or more cloud-based platform(s) such as MICROSOFT AZURE, AMAZON AWS, or the like. Server(s) 302 may include one or more processor(s) 304 (i.e., CPU(s)) as well as one or more computer memories 306.

Memories 306 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memorie(s) 306 may store an operating system (OS) (e.g., Microsoft Windows, Linux, UNIX, etc.) capable of facilitating the functionalities, apps, methods, or other software as discussed herein. Memorie(s) 306 may also store an artificial intelligence (AI) model 308, which may comprise an artificial intelligence-based model, such as a machine learning model, trained on data (e.g., biometric signal data), as described herein. Additionally, or alternatively, the AI model 308 may also be stored in database 305, which is accessible or otherwise communicatively coupled to server(s) 302. In addition, memories 306 may also store machine readable instructions, including any of one or more application(s) (e.g., an imaging application as described herein), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For example, at least some of the applications, software components, or APIs may be, include, otherwise be part of, a machine learning model or component, such as the AI model 308, where each may be configured to facilitate their various functionalities discussed herein. It should be appreciated that one or more other applications may be envisioned and that are executed by the processor(s) 304.

The processor(s) 304 may be connected to the memories 306 via a computer bus responsible for transmitting electronic data, data packets, or otherwise electronic signals to and from the processor(s) 304 and memories 306 in order to implement or perform the machine-readable instructions, methods, processes, elements or limitations, as illustrated, depicted, or described for the various flowcharts, illustrations, diagrams, figures, and/or other disclosure herein.

Processor(s) 304 may interface with memory 306 via the computer bus to execute an operating system (OS). Processor(s) 304 may also interface with the memory 306 via the computer bus to create, read, update, delete, or otherwise access or interact with the data stored in memories 306 and/or the database 305 (e.g., a relational database, such as Oracle, DB2, MySQL, or a NoSQL based database, such as MongoDB). The data stored in memories 306 and/or database 305 may include all or part of any of the data or information described herein, including, for example, biometric signal data, visualizations, user settings, or any other data as described herein.

Server(s) 302 may further include a communication component configured to communicate (e.g., send and receive) data via one or more external/network port(s) to one or more networks or local terminals, such as computer network 320 and/or terminal 309 as described herein. In some embodiments, server(s) 302 may include a client-server platform technology such as ASP. NET, Java J2EE, Ruby on Rails, Node. js, a web service or online API, responsive for receiving and responding to electronic requests. The server(s) 302 may implement the client-server platform technology that may interact, via the computer bus, with the memories(s) 306 (including the applications(s), component(s), API(s), data, etc. stored therein) and/or database 305 to implement or perform the machine readable instructions, methods, processes, elements or limitations, as illustrated, depicted, or described for the various flowcharts, illustrations, diagrams, figures, and/or other disclosure herein.

In various embodiments, the server(s) 302 may include, or interact with, one or more transceivers (e.g., WWAN, WLAN, and/or WPAN transceivers) functioning in accordance with IEEE standards, 3GPP standards, or other standards, and that may be used in receipt and transmission of data via external/network ports connected to computer network 320. In some embodiments, computer network 320 may comprise a private network or local area network (LAN). Additionally, or alternatively, computer network 320 may comprise a public network such as the Internet.

Server(s) 302 may further include or implement an operator interface configured to present information to an administrator or operator and/or receive inputs from the administrator or operator. As shown in FIG. 3, an operator interface may provide a display screen (e.g., via terminal 309). Server(s) 302 may also provide I/O components (e.g., ports, capacitive or resistive touch sensitive input panels, keys, buttons, lights, LEDs), which may be directly accessible via, or attached to, server(s) 302 or may be indirectly accessible via or attached to terminal 309. According to some embodiments, an administrator or operator may access server 302 via terminal 309 to review information, make changes, input training data or images, initiate training of AI model 308, and/or perform other functions.

In general, a computer program or computer based product, application, or code (e.g., the model(s), such as AI models, or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 304 (e.g., working in connection with the respective operating system in memories 306) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C #, Objective-C, Java, Scala, ActionScript, JavaScript, HTML, CSS, XML, etc.).

As shown in FIG. 3, server(s) 302 are communicatively connected, via computer network 320 to the one or more computing devices 311c1-311c3 and/or 312c1-312c3 via base stations 311b and 312b, respectively. Each of computing devices 311c1-311c3 and/or 312c1-312c3 may comprise a computing device 111 as described herein for FIG. 1.

In some embodiments, base stations 311b and 312b may comprise cellular base stations, such as cell towers, communicating to the one or more computing devices 311c1-311c3 and 312c1-312c3 via wireless communications 321 based on any one or more of various mobile phone standards, including NMT, GSM, CDMA, UMMTS, LTE, 5G, or the like. Additionally, or alternatively, base stations 311b and 312b may comprise routers, wireless switches, or other such wireless connection points communicating to the one or more computing devices 311c1-311c3 and 312c1-312c3 via wireless communications 322 based on any one or more of various wireless standards, including by non-limiting example, IEEE 802.11a/b/c/g (WIFI), the BLUETOOTH standard, or the like.

Any of the one or more computing devices 311c1-311c3 may comprise mobile devices and/or client devices for accessing and/or communications with server(s) 302. Such mobile devices may comprise one or more mobile processor(s) and/or an imaging device for capturing images displaying video, AR, VR, etc. as described herein. In various embodiments, computing devices 311c1-311c3 and/or 312c1-312c3 may comprise a mobile phone (e.g., a cellular phone), a tablet device, a personal data assistance (PDA), or the like, including, by non-limiting example, an APPLE iPhone or iPad device or a GOOGLE ANDROID based mobile phone or table.

In various embodiments, the one or more computing devices 311c1-311c3 may implement or execute an operating system (OS) or mobile platform such as Apple's iOS and/or Google's Android operation system. Any of the one or more computing devices 311c1-311c3 may comprise one or more processors and/or one or more memories for storing, implementing, or executing computing instructions or code, e.g., a mobile application or a home or personal assistant application, as described in various embodiments herein. As shown in FIG. 3, AI model 308a and/or a software application as described herein, or at least portions thereof, may also be stored locally on a memory of a computing device (e.g., computing device 311c1). In some aspects, AI model 308a as installed on a computing device may comprise the same AI model 308 as installed on server(s) 302. Additionally, or alternatively, AI model 308a may comprise a portion of AI model 308 as installed on server(s) 302. It is to be understood that in some aspects, the AI model may be installed wholly at computing device, wholly at server(s) 302, or partially on computing device and partially on server(s) 302 where communication between AI model 308a and AI model 308 occurs through computer network 320. Generally, when AI learning model is referred to herein, it refers to one or both of AI model 308 and/or based learning model 108a.

Computing devices 311c1-311c3 may comprise a wireless transceiver to receive and transmit wireless communications 321 and/or 322 to and from base stations 311b and/or 312b. In various embodiments, biometric signal data, e.g., as collected by one or more biosensors of sensor pod(s) as described for FIGS. 1A and/or 1B, may be transmitted via computer network 320 to server(s) 302 for training of model(s) (e.g., AI model 308) and/or performing analysis, generation, and/or visualization as described herein, including, for example, as described herein for FIG. 4.

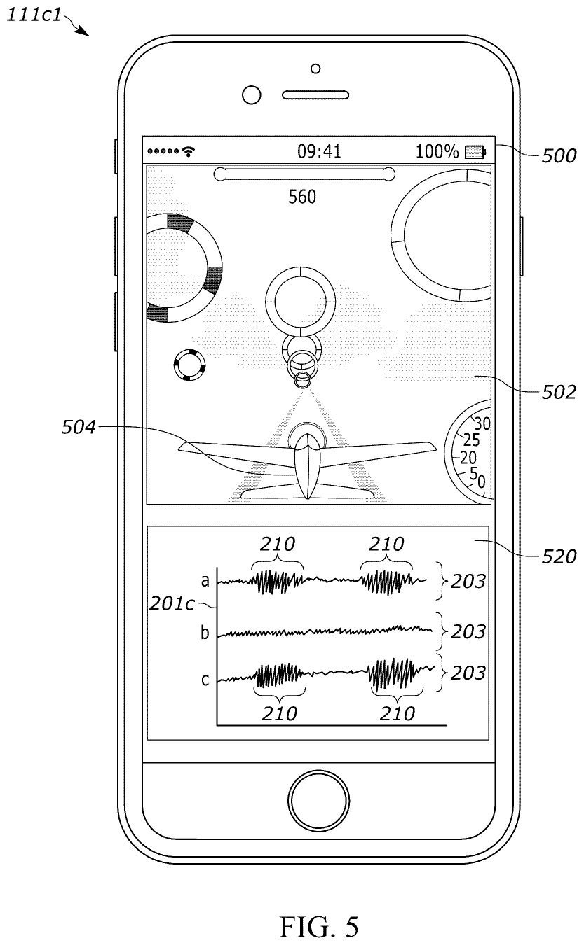

In addition, each of the one or more user computer devices 311c1-311c3 may include a display screen for displaying graphics, images, text, visualizing a virtual limb, visually controlling a graphical aspect, visualizing the one or more user-specific biosignal patterns on a display device, and/or other such visualizations or information as described herein. In various embodiments, graphics, images, text, visualizations, and/or other such visualizations or information may be received from server(s) 302 for display on the display screen of any one or more of user computer devices 311c1-311c3. Additionally, or alternatively, a user computer device, e.g., as described herein for FIG. 5, may comprise, implement, have access to, render, or otherwise expose, at least in part, an interface or a guided user interface (GUI) for displaying text, images, visualizations on its display screen.

In some embodiments, computing instructions and/or applications executing at the server (e.g., server(s) 302) and/or at a mobile device (e.g., mobile device 311c1) may be communicatively connected for detecting intended limb motion and invoke pain mitigation, as described herein. For example, one or more processors (e.g., processor(s) 304) of server(s) 302 may be communicatively coupled to a mobile device via a computer network (e.g., computer network 320). In such embodiments, an imaging app may comprise a server app portion configured to execute on the one or more processors of the server (e.g., server(s) 302) and a mobile app portion configured to execute on one or more processors of the mobile device (e.g., any of one or more computing devices 311c1-311c3 and/or 312c1-312c3). In such embodiments, the server app portion is configured to communicate with the mobile app portion. The server app portion or the mobile app portion may each be configured to implement, or partially implement, one or more of: (1) sensing, by one or more biosensors, biometric signal data of a user while the user performs an intended limb motion of a given limb of the user; (2); analyzing, by one or more processors, the biometric signal data to learn in real time or near real time one or more user-specific biosensor patterns detected when the user performs the intended limb motion; (3) generating, by one or more processors, based on the user-specific limb motion intent, learned user intent data; and/or (4) visualizing, by one or more processors, the learned user intent data on the display device for invoking a pain mitigation effect with the user corresponding to the given limb.

As shown in the example of FIG. 3, mobile device 311c1 may correspond to computing device 111 of FIG. 1, where biometric wearable device 102 of user 101 can transmit and receive biometric signal data, settings, visualizations to and from server 302 via network 320. Similarly, each of mobile device 312c1 may correspond to a computing device (e.g., a computing device 111 of FIG. 1), where respective biometric wearable devices (e.g., a biometric wearable device 102) of user 152 and user 153 can transmit and receive biometric signal data, settings, visualizations to and from server 302 via network 320.

In various aspects, the biometric wearable device 102 may send data to the server(s) of cloud-based network 300. Such data may be transmitted to and from a server remote (e.g., a cloud-based server, e.g., server(s) 302) to the biometric wearable device 102. In various aspects, such data may comprise myoelectric data, settings, gameplay scores, usage, etc.

For example, transceiver 102t may transmit via computer network 320 the biometric signal data of the user to server(s) 302. In various aspects, the biometric signal data of the user (and/or various users such as 101, 152, and/or 153) may be stored in database 305. Such data may be used for training AI model 308 and/or AI model 308a, for example, as described herein.

Additionally, or alternatively, transceiver 102t may transmit via computer network 320 one or more user-specific biosignal patterns of the user to server(s) 302. Such user-specific biosignal patterns may comprise, for example, any of the patterns of the user when performing a limb motion or otherwise articulating or moving his other limb. Such patterns may include patters of biometric signal data, such as shown, by way of non-limiting example, for FIGS. 2A-2C herein.

Additionally, or alternatively, transceiver 102t may transmit via computer network 320 learned user intent data to server(s) 302. The learned user intent data may comprise images or visualizations viewed by the user and/or may also comprise positions (e.g., as determined by pixels) within images for mapping or correlating the user's intended limb motion within a given image, frame, or visualization, where such images, visualization, positions, or otherwise data, and can be stored in database 305 and/or can be used, for example, for training AI model 308 and/or AI model 308a.

Additionally, or alternatively, transceiver 102t may transmit via computer network 320 data associated with the biometric signal data of the user to server(s) 302. Such data may comprise, by way of non-limiting example, settings of the user, including for example, settings of the biometric wearable device 102 of the user. Further, such data may include settings for specific games, visualization tools or applications, or the like for customizing or making user-specific a pain mitigation effect within a given visualization tool or application that the user can launch or otherwise execute from a menu of an interface, e.g., user interface 121u and/or user interface device 121ud.

In various aspects, any one or more of the above-mentioned data, e.g., as transmitted by transceiver 102t to server(s) 302 or otherwise as described herein, may be provided to an artificial intelligence (AI) algorithm for training or retraining an AI model (e.g., AI model 308 and/or AI model 308a). In various aspects, an AI model (e.g., such as AI model 308 and/or AI model 308a) is trained or otherwise configured to analyze biometric signal data (e.g., biometric wearable device 102) to identify one or more user-specific biosignal patterns and/or user-specific limb motion intent as detected when the user performs the intended limb motion. The one or more user-specific biosignal patterns and/or user-specific limb motion intent may be those described herein, by way of non-limiting example, for FIGS. 2A-2C. The identification of such one or more user-specific biosignal patterns may be executed or performed in real time or near real time.

In various aspects with respect to AI modeling, one or more processor(s) (e.g., processor 304) of cloud-based computing platform (e.g., server(s) 302) may receive data, e.g., the biometric signal data, learned user intent data, biosignal patterns data, and/or other data as described herein, for a plurality of individuals (e.g., users 101, 152, and/or 153) via a computer network (e.g., computer network 320). In various embodiments, a machine learning imaging model, as described herein (e.g. AI model 308), may be trained using a supervised or unsupervised machine learning program or algorithm. The machine learning program or algorithm may employ a neural network, which may be a convolutional neural network, a vision transformer, a deep learning neural network, or a combined learning module or program that learns in two or more features or feature datasets (e.g., biometric signal data, pattern data, and/or positioning data for biometric visualizations). The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques. In some embodiments, the artificial intelligence and/or machine learning based algorithms may be included as a library or package executed on server(s) 302. For example, libraries may include the TENSORFLOW based library, the PYTORCH library, the SCIKIT-LEARN Python library, and/or other library as accessible by the HUGGING FACE library.

Machine learning model(s), such as an AI learning model as described herein for some embodiments, may be created and trained based upon example data (e.g., “training data” and related pixel data) inputs or data (which may be termed “features” and “labels”) in order to make valid and reliable predictions for new inputs, such as testing level or production level data or inputs. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output.

In unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated.

Supervised learning and/or unsupervised machine learning may also comprise retraining, relearning, or otherwise updating models with new, or different, information, which may include information received, ingested, generated, or otherwise used over time. The disclosures herein may use one or both of such supervised or unsupervised machine learning techniques.

In various embodiments, training an AI model (e.g., AI model 308 and/or AI model 308a) machine learning may involve identifying and recognizing patterns in existing data (such as identifying features in biometric signal data, learned user intent data, biosignal patterns data, and/or other data as described herein in order to facilitate output of predictions, classifications, or otherwise values (e.g., a dependent variable). For example, an AI model may be trained, when provided with new data (e.g., biometric signal data) as input, to output a prediction or classification of one or more user-specific biosignal patterns as detected when the user performs the intended limb motion. For example, the prediction may comprise a value, within a certain percentage or degree of accuracy, for detecting when a user has performed a given limb motion (e.g., moving arm up or down, waiving, signaling with certain fingers, etc.). A classification may comprise determine which class or otherwise type of limb motion was performed (e.g.., moving arm up or down, waiving, signaling with certain fingers, etc.). In this way, a biometric wearable device 102 may sense a user's biometric signal data to detect a limb motion or gesture of the user.

FIG. 4 is a block diagram illustrating an example biometric wearable method 400 for detecting intended limb motion and invoking pain mitigation, in accordance with various aspects herein. At block 410, method 400 comprises sensing, by one or more biosensors (e.g., biosensor 102sen), biometric signal data of a user. The biometric signal data may comprise data sensed or otherwise collected while the user performs an intended limb motion (e.g., rotating) of a given limb (e.g., an arm) of the user (e.g., user 101).

In various aspects, each of the one or more biosensors (e.g., biosensor 102sen and/or other biosensors shown for FIG. 1A or 1B) form part of one or more sensor pods of a biometric wearable device (e.g., biometric wearable device 102) worn by the user (e.g., user 101). Each the one or more sensor pods may include one or more biofeedback indicators (e.g., LEDs, LCD display(s), vibrotactile elements, speakers, buzzers, or the like).

At block 420, method 400 comprises analyzing, by one or more processors (e.g., processor 111p, processor 102p, and/or processor 304), the biometric signal data to learn in real time or near real time a user-specific limb motion intent based on one or more user-specific biosignal patterns detected when the user performs the intended limb motion.

In some aspects, the analysis may call, execute, or invoke an AI model. For example, an AI model (e.g., AI model 308 and/or AI model 308a) may be implemented where the AI model is provided biometric signal data as input. The AI model would have been previously trained on biometric signal data for outputting prediction(s) and/or classification(s) and/or classifications of various user(s) intended limb motions. In the instant example, the AI model can take the user-specific biometric signal data to output a prediction and/or classification. The prediction and/or classification is based on the one or more user-specific biosignal patterns detected by the AI model when the user performs the intended limb motion. Such detection can occur in real time or near real time as the user performs the intended limb motion, and where the AI model receives as input, in real time or near real time, the respective user-specific biometric signal information. The AI model's output (e.g., predictions and/or classifications based the detected biosignal patterns) can be provided the one or more processors to learn in real time or near real time a user-specific limb motion intent and/or for visualization, control, or other use as described herein.

At block 430, method 400 comprises generating, by the one or more processors (e.g., processor 111p, processor 102p, and/or processor 304) based on the one or more user-specific biosignal patterns and/or user-specific limb motion intent, learned user intent data. The learned user intent data may be generated or otherwise configured for various display, visualization, and/or control purposes on a display device to provide feedback and/or information to the user. For example, the learned user intent data may comprise data for visualizing a virtual limb (e.g., an arm) on a display device (e.g., user interface device 121ud). Additionally, or alternatively, the learned user intent data may comprise data for visually controlling a graphical aspect, e.g., such as virtual limb, computer graphic, video game character or limb thereof or other graphical feature, e.g., as described herein for FIG. 5. Still further, additionally, or alternatively, the learned user intent data may comprise data for visualizing the one or more user-specific biosignal patterns (e.g., as shown for FIGS. 2A-2C and/or FIG. 5) on a display device. Still further, the learned user intent data may comprise visualizing the user-specific limb motion intent data on the display device. Still further, learned user intent data can be used to control output of the display device based on the user-specific limb motion intent.

Further, in various aspects the learned user intent data may also be configured to display a suite of visualization tools to produce pain mitigation. The visualization tools could include a suite of programs launchable from the display or interface, and could include, for example, interactive games, different display views and formats of biometric signal data, or user customized activities that could have the user move his or her limb in a manner to invoke of pain mitigation. In one example, a user (e.g., user 101) could launch an application for playing a biometric signal-based game, which causes the user to use his or her limb in a particular manner (e.g., invoke a muscle bulge) in a way to cause a physical response for reducing pain.

In another example, a game may include a graphical aspect or video frames where the user (e.g., user 101) is flying in or on an on-screen airplane through targets, for example, as shown for FIG. 5. Such activity of the plane is controlled by the pattern recognition of biometric signal data as sensed by biometric wearable device 102. In the example of FIG. 5, biometric sensor but data is displayed (e.g., as sensed by biometric wearable device 102 and as described for FIG. 2C herein)

At block 440, method 400 comprises visualizing, by the one or more processors (e.g., processor 111p, processor 102p, and/or processor 304), the learned user intent data on the display device for invoking a pain mitigation effect with the user corresponding to the given limb. In various aspects, the pain mitigation effect reduces at least one ailment as experienced by the user. The ailment may comprise a medical or other condition such as limb pain, phantom limb pain, complex regional pain syndrome (CRPS), post-traumatic stress disorder (PTSD), and/or burning sensation(s).

In some aspects, the display device that displays the learned user intent data. may comprise a screen device (e.g., a mobile device or user interface 121u) or a headset device (e.g., a VR and/or AR device such as user interface device 121ud). In such aspects, the virtual limb or the one or more user-specific biosignal patterns and/or specific limb motion intent data can be visualized in a virtual environment, e.g., such as via avatar movement, gameplay actions, visualizations, or as otherwise described herein.

Additionally, or alternatively, the display device may comprise a display screen connected to the biometric wearable device (e.g., biometric wearable device 102). In such aspects, the virtual limb (e.g., virtual limb 117ev) or one or more user-specific biosignal patterns and/or specific limb motion intent data can be visualized on the display screen, which can be a display screen built into or on the biometric wearable device 102 itself.

In various aspects, various visualizations, such as those described for generated learned user intent data, can be displayed or otherwise visualized simultaneously. In one example, the feedback (e.g., biosignal data) from the wearable device is displayed for a pain mitigation benefit or effect in one or a combination of outputs: (1) the user is displayed the biosignal themselves (e.g., before such signals are algorithmically applied or analyzed by the processor or otherwise a decoder used by the processor to analyze the signals), and/or (2) the user is displayed user-specific limb motion intent, as produced by the processor(s) (e.g., implementing a decoding (pattern recognition) algorithm of the biosignal). That is, some users experience pain mitigation from seeing the input (e.g., the biosignal patterns themselves) signals, and others benefit from seeing the output (e.g., a visualization of the user-specific limb motion intent), and some benefit from both. In various aspects, the computing instructions herein (e.g., mobile app) allow a user to display, access, set, or otherwise access both such display types, at will, where such display types can be visualized simultaneously.

FIG. 5 illustrates an example user interface 502 as rendered on a display screen 500 of a computing device (e.g., user computing device 311c1 and/or computing device 111) in accordance with various embodiments disclosed herein. For example, as shown in the example of FIG. 5, user interface 502 may be implemented or rendered via an application (app) executing on user computing device 311c1. User interface 502 may be implemented or rendered via a native app executing on user computing device 311c1. In the example of FIG. 5, user computing device 311c1 is a user computer device as described for FIG. 3, e.g., where 311c1 is illustrated as an APPLE iPhone that implements the APPLE iOS operating system and that has display screen 500. User computing device 311c1 may execute one or more native applications (apps) on its operating system, including, for example, imaging app as described herein. Such native apps may be implemented or coded (e.g., as computing instructions) in a computing language (e.g., SWIFT) executable by the user computing device operating system (e.g., APPLE iOS) by the processor of user computing device 311c1.

Additionally, or alternatively, user interface 502 may be implemented or rendered via a web interface, such as via a web browser application, e.g., Safari and/or Google Chrome app(s), or other such web browser or the like.

As shown in the example of FIG. 5, user interface 502 comprises a window 502 depicting learned user intent data comprising visualization and/or control of one or more graphical aspects in a video game. The video game may have been launched by a visualization tool or application within an interface (e.g., user interface 121u and/or user interface device 121ud). As shown, the video game of window 502 depicts a flight simulator game that may comprise an on-screen airplane 504 controlled by the user through a target 506 and/or additional targets as shown for window 502. Window 502 further comprises textual renderings regarding information pertinent to the video game, including a score 560 and/or a speed 510 of the aircraft. The values of the score 560 and/or speed 510 changes as the user interacts with the video game.

In the example, airplane 504 may be controlled by the user's limb motion(s) where biometric wearable device 102 may sense the user's biometric signal data and analyze such biometric signal data to detect biosignal patterns. In some aspects, an AI model (e.g., AI model 308 and/or AI model 308a) may detect the biosignal patterns and output a prediction and/or classification identifying one or more specific intended limb motion(s) of the user. In various aspects, the analysis, including the predictions, classification, and/or otherwise output, may be used to control a graphical aspect (e.g., motion of the airplane 504) in real time or near real time within the video game of window 502. Control of the graphical aspect can be implemented by receiving the user's biometric signal data and mapping or correlating the user's intended limb motion(s) within a given image, frame, or visualization (e.g., airplane 504). Such images, visualization, positions, or otherwise data can be controlled based on the mapping. For example, a biometric signal data and/or user-specific biosignal patterns of data may indicate that the user wishes to perform a certain action, such as moving airplane 504 through a target (e.g., target 506). The mapping can comprise assigning an object or graphic within the video game a position within the game, e.g., a center or bottom of the window 502, where the biometric signal data and/or user-specific biosignal patterns can be input to the video game as input to control airplane 502 within the space of window 502.

In various aspects, the control of the graphical aspect (e.g., airplane 504) and/or the changing of the values (e.g., score 560 and speed 510) can provide a source of pain mitigation because control of such aspects invokes pain reduction feedback for the user.

In addition, as shown for FIG. 5, window 520 illustrates an example of learned user intent data comprising visualizing the one or more biometric data and/or signals and/or user-specific biosignal patterns on a display device. The one or more the one or more biometric data and/or signals and/or user-specific biosignal patterns can be those as described in FIGS. 2A-2C. In the example of FIG. 5, the one or more biometric data and/or signals and/or user-specific biosignal patterns are those as shown for FIG. 2C. Such data comprise data for visualizing the one or more user-specific biosignal patterns and/or user-specific limb motion intent, on a display device via a display screen, e.g., display screen 500.

In various aspects, the display of the one or more biometric data and/or signals and/or user-specific biosignal patterns can provide a source of pain mitigation because display of such data can invoke pain reduction feedback for the user.

In various embodiments, the graphical aspects, visualizations, text, and/or biometric data and/or signals and/or user-specific biosignal patterns may be transmitted via the computer network 320, from server(s) 302, to the user computing device of the user for rendering on the display screen 500 of the user computing device (e.g., computing device 311c1).

In other embodiments, no transmission to the server(s) 302 occurs, where the graphical aspects, visualizations, text, and/or biometric data and/or signals and/or user-specific biosignal patterns may instead be generated locally, by AI model (e.g., AI model 308a) executing and/or implemented on the user's mobile device (e.g., computing device 311c1) and rendered, by a processor of the mobile device, on a display screen of the mobile device.

### Aspects of the Disclosure

The following aspects are provided as examples in accordance with the disclosure herein and are not intended to limit the scope of the disclosure.

Aspect 1. A biometric wearable system configured to detect intended limb motion and invoke pain mitigation, the biometric wearable system comprising: a biometric wearable device comprising plurality of sensor pods, each sensor pod of the plurality of sensor pods including one or more biosensors configured to sense biometric signal data of a user, and further including one or more biofeedback indicators; one or more processors communicatively coupled to the one or more biosensors; a memory storing computing instructions that when executed by the one or more processors to cause the one or more processors to: sense the biometric signal data of the user from the one or more biosensors while the user performs an intended limb motion of a given limb of the user, analyze the biometric signal data to learn in real time or near real time a user-specific limb motion intent based on one or more user-specific biosignal patterns detected when the user performs the intended limb motion, generate, based on the user-specific limb motion intent, learned user intent data for at least one of: (1) visualizing a virtual limb on a display device, (2) visually controlling a graphical aspect on the display device, (3) visualizing the one or more user-specific biosignal patterns on the display device, and/or (4) visualizing the user-specific limb motion intent data on the display device, and visualize the learned user intent data on the display device for invoking a pain mitigation effect with the user corresponding to the given limb.

Aspect 2. The biometric wearable system of aspect 1, wherein the pain mitigation effect reduces at least one ailment as experienced by the user comprising: (a) limb pain, (b) phantom limb pain, (c) complex regional pain syndrome (CRPS), (d) post-traumatic stress disorder (PTSD), or (e) burning sensation.

Aspect 3. The biometric wearable system of any one of the preceding aspects, wherein the display device is a screen device or a headset device.

Aspect 4. The biometric wearable system of any one of the preceding aspects further comprising: a transceiver communicatively coupled to the one or more processors, wherein the learned user intent data is transmitted via the transceiver to the display device.

Aspect 5. The biometric wearable system of any one of the preceding aspects, wherein the virtual limb or the one or more user-specific biosignal patterns and/or specific limb motion intent data is visualized in a virtual environment.

Aspect 6. The biometric wearable system of any one of the preceding aspects, wherein the display device is a display screen connected to the biometric wearable device, and wherein the virtual limb or one or more user-specific biosignal patterns and/or specific limb motion intent data is visualized on the display screen.

Aspect 7. The biometric wearable system of any one of the preceding aspects, wherein the computing instructions, when executed by the one or more processors further cause the one or more processors to implement at least one of: receive an input from a computing device to configure or alter an operation or setting of the biometric wearable device; and/or provide an indication of an operation or status of the biometric wearable device via at least one of the one or more biofeedback indicators of at least one of the plurality of sensor pods.

Aspect 8. The biometric wearable system of any one of the preceding aspects, wherein the plurality of sensor pods is part of a wearable prosthetic liner interface and/or a partial sleeve interface.

Aspect 9. The biometric wearable system of any one of the preceding aspects, wherein the one or more biofeedback indicators of each sensor pod are configured to provide an output to indicate amplitude information of a signal detected by the one or more biosensors of the sensor pods.

Aspect 10. The biometric wearable system of aspect 9, wherein the output of the one or more biofeedback indicators of each sensor pod comprises an illumination, in a predetermined pattern, to indicate amplitude information of a signal detected by the one or more biosensors of the sensor pods.

Aspect 11. The biometric wearable system of aspect 9, wherein the intended limb motion comprises a muscle bulge of the given limb of the user causing an increase of pressure of the given limb against the plurality of sensor pods, wherein the increased pressure invokes a sensorimotor effect that enhances the pain mitigation effect.

Aspect 12. The biometric wearable system of aspect 11, wherein the increase in muscle bulge causes a corresponding increase in the user's biosignal output causing at least one of: (1) an illumination of a plurality of light emitting diodes of at least one sensor pod of the plurality of sensor pods, and/or (2) a frequency or vibration of actuators of at least one sensor pod of the plurality of sensor pods, wherein the increased pressure and the increase in the output invokes a sensorimotor effect that enhances the pain mitigation effect.

Aspect 13. The biometric wearable system of any one of the preceding aspects further comprising a transceiver, wherein data comprising any one or more of: (1) the biometric signal data of the user, (2) the one or more user-specific biosignal patterns and/or a user-specific limb motion intent of the user, and/or (3) the learned user intent data, and/or (4) data associated with the biometric signal data of the user, is configured to be transmitted to and from a server remote to the biometric wearable device.

Aspect 14. The biometric wearable system of aspect 13, wherein the data provided to an artificial intelligence (AI) algorithm for training or retraining an AI model configured to analyze the biometric signal data to identify in real time or near real time the one or more user-specific biosignal patterns and/or a user-specific limb motion intent as detected when the user performs the intended limb motion.

Aspect 15. A biometric wearable method for detecting intended limb motion and invoking pain mitigation, the biometric wearable method comprising: sensing, by one or more biosensors, biometric signal data of a user while the user performs an intended limb motion of a given limb of the user, wherein each of the one or more biosensors form part of one or more sensor pods of a biometric wearable device worn by the user, and wherein each the one or more sensor pods includes one or more biofeedback indicators; analyzing, by one or more processors, the biometric signal data to learn in real time or near real time a user-specific limb motion intent based on one or more user-specific biosignal patterns detected when the user performs the intended limb motion; generating, by the one or more processors based on the user-specific limb motion intent, learned user intent data for at least one of: (1) visualizing a virtual limb on a display device, (2) visually controlling a graphical aspect on the display device, (3) visualizing the one or more user-specific biosignal patterns on the display device, and/or (4) visualizing the user-specific limb motion intent data on the display device; and visualizing, by the one or more processors, the learned user intent data on the display device for invoking a pain mitigation effect with the user corresponding to the given limb.

Aspect 16. The biometric wearable method of aspect 15, wherein the pain mitigation effect reduces at least one ailment as experienced by the user comprising: (a) limb pain, (b) phantom limb pain, (c) complex regional pain syndrome (CRPS), (d) post-traumatic stress disorder (PTSD), or (e) burning sensation.

Aspect 17. The biometric wearable method of any one of aspects 15-16, wherein the display device is a screen device or a headset device.

Aspect 18. The biometric wearable method of any one of aspects 15-17 further comprising transmitting the learned user intent data via a transceiver to the display device.

Aspect 19. The biometric wearable method of any one of aspects 15-18, wherein the virtual limb or the one or more user-specific biosignal patterns and/or specific limb motion intent data is visualized in a virtual environment.

Aspect 20. The biometric wearable method of any one of aspects 15-19, wherein the display device is a display screen connected to the biometric wearable device, and wherein the virtual limb or one or more user-specific biosignal patterns and/or specific limb motion intent data is visualized on the display screen.

Aspect 21. The biometric wearable method of any one of aspects 15-20 further comprising: receiving an input from a computing device to configure or alter an operation or setting of the biometric wearable device; and/or providing an indication of an operation or status of the biometric wearable device via at least one of the one or more biofeedback indicators of at least one of the one or more sensor pods.

Aspect 22. The biometric wearable method of any one of aspects 15-21, wherein the one or more sensor pods is part of a wearable prosthetic liner interface and/or a partial sleeve interface.

Aspect 23. The biometric wearable method of any one of aspects 15-22, wherein the one or more biofeedback indicators of each sensor pod are configured to provide an output to indicate amplitude information of a signal detected by the one or more biosensors of the sensor pods.

Aspect 24. The biometric wearable method of aspect 23, wherein the output of the one or more biofeedback indicators of each sensor pod comprises an illumination, in a predetermined pattern, to indicate amplitude information of a signal detected by the one or more biosensors of the sensor pods.

Aspect 25. The biometric wearable method of aspect 23, wherein the intended limb motion comprises a muscle bulge of the given limb of the user causing an increase of pressure of the given limb against the one or more sensor pods, wherein the increased pressure invokes a sensorimotor effect that enhances the pain mitigation effect.

Aspect 26. The biometric wearable method of aspect 25, wherein the increase in muscle bulge causes a corresponding increase in the user's biosignal output causing at least one of: (1) an illumination of a plurality of light emitting diodes of at least one sensor pod of the plurality of sensor pods, and/or (2) a frequency or vibration of actuators of at least one sensor pod of the plurality of sensor pods, wherein the increased pressure and the increase in the output invokes a sensorimotor effect that enhances the pain mitigation effect.

Aspect 27. The biometric wearable method of aspect 15 further comprising transmitting via a transceiver, to and from a server remote to the biometric wearable device, data comprising any one or more of: (1) the biometric signal data of the user, (2) the one or more user-specific biosignal patterns and/or a user-specific limb motion intent of the user, and/or (3) the learned user intent data, and/or (4) data associated with the biometric signal data of the user, is configured to be transmitted to and from a server remote to the biometric wearable device.

Aspect 28. The biometric wearable method of aspect 27 further comprising providing the server to an artificial intelligence (AI) algorithm for training or retraining an AI model configured to analyze the biometric signal data to identify in real time or near real time the one or more user-specific biosignal patterns and/or a user-specific limb motion intent as detected when the user performs the intended limb motion.

Aspect 29. A tangible, non-transitory computer-readable medium storing instructions for detecting intended limb motion and invoking pain mitigation, that when executed by one or more processors cause the one or more processors to: sense, by one or more biosensors, biometric signal data of a user while the user performs an intended limb motion of a given limb of the user, wherein each of the one or more biosensors form part of one or more sensor pods of a biometric wearable device worn by the user, and wherein each the one or more sensor pods includes one or more biofeedback indicators; analyze, by one or more processors, the biometric signal data to learn in real time or near real time a user-specific limb motion intent based on one or more user-specific biosignal patterns detected when the user performs the intended limb motion; generate, by the one or more processors based on the user-specific limb motion intent, learned user intent data for at least one of: (1) visualizing a virtual limb on a display device, (2) visually controlling a graphical aspect on the display device, (3) visualizing the one or more user-specific biosignal patterns on the display device, and/or (4) visualizing the user-specific limb motion intent data on the display device; and visualize, by the one or more processors, the learned user intent data on the display device for invoking a pain mitigation effect with the user corresponding to the given limb.

### Additional Disclosure

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location, while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other embodiments, the one or more processors or processor-implemented modules may be distributed across a number of geographic locations.

This detailed description is to be construed as exemplary only and does not describe every possible embodiment, as describing every possible embodiment would be impractical, if not impossible. A person of ordinary skill in the art may implement numerous alternate embodiments, using either current technology or technology developed after the filing date of this application.

Those of ordinary skill in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed is:

1. A biometric wearable system configured to detect intended limb motion and invoke pain mitigation, the biometric wearable system comprising:
a biometric wearable device comprising plurality of sensor pods, each sensor pod of the plurality of sensor pods including one or more biosensors configured to sense biometric signal data of a user, and further including one or more biofeedback indicators;
one or more processors communicatively coupled to the one or more biosensors;
a memory storing computing instructions that when executed by the one or more processors to cause the one or more processors to:
sense the biometric signal data of the user from the one or more biosensors while the user performs an intended limb motion of a given limb of the user,
analyze the biometric signal data to learn in real time or near real time a user-specific limb motion intent based on one or more user-specific biosignal patterns detected when the user performs the intended limb motion, generate, based on the user-specific limb motion intent, learned user intent data for at least one of: (1) visualizing a virtual limb on a display device, (2) visually controlling a graphical aspect on the display device, (3) visualizing the one or more user-specific biosignal patterns on the display device, and/or (4) visualizing the user-specific limb motion intent data on the display device, and
visualize the learned user intent data on the display device for invoking a pain mitigation effect with the user corresponding to the given limb.

2. The biometric wearable system of claim 1, wherein the pain mitigation effect reduces at least one ailment as experienced by the user comprising: (a) limb pain, (b) phantom limb pain, (c) complex regional pain syndrome (CRPS), (d) post-traumatic stress disorder (PTSD), or (e) burning sensation.

3. The biometric wearable system of claim 1, wherein the display device is a screen device or a headset device.

4. The biometric wearable system of claim 1 further comprising:
a transceiver communicatively coupled to the one or more processors,
wherein the learned user intent data is transmitted via the transceiver to the display device.

5. The biometric wearable system of claim 1, wherein the virtual limb or the one or more user-specific biosignal patterns and/or specific limb motion intent data is visualized in a virtual environment.

6. The biometric wearable system of claim 1, wherein the display device is a display screen connected to the biometric wearable device, and wherein the virtual limb or one or more user-specific biosignal patterns and/or specific limb motion intent data is visualized on the display screen.

7. The biometric wearable system of claim 1, wherein the computing instructions, when executed by the one or more processors further cause the one or more processors to implement at least one of:
receive an input from a computing device to configure or alter an operation or setting of the biometric wearable device; and/or
provide an indication of an operation or status of the biometric wearable device via at least one of the one or more biofeedback indicators of at least one of the plurality of sensor pods.

8. The biometric wearable system of claim 1, wherein the plurality of sensor pods is part of a wearable prosthetic liner interface and/or a partial sleeve interface.

9. The biometric wearable system of claim 1, wherein the one or more biofeedback indicators of each sensor pod are configured to provide an output to indicate amplitude information of a signal detected by the one or more biosensors of the sensor pods.

10. The biometric wearable system of claim 9, wherein the output of the one or more biofeedback indicators of each sensor pod comprises an illumination, in a predetermined pattern, to indicate amplitude information of a signal detected by the one or more biosensors of the sensor pods.

11. The biometric wearable system of claim 9, wherein the intended limb motion comprises a muscle bulge of the given limb of the user causing an increase of pressure of the given limb against the plurality of sensor pods, wherein the increased pressure invokes a sensorimotor effect that enhances the pain mitigation effect.

12. The biometric wearable system of claim 11, wherein the increase in muscle bulge causes a corresponding increase in the user's biosignal output causing at least one of: (1) an illumination of a plurality of light emitting diodes of at least one sensor pod of the plurality of sensor pods, and/or (2) a frequency or vibration of actuators of at least one sensor pod of the plurality of sensor pods, wherein the increased pressure and the increase in the output invokes a sensorimotor effect that enhances the pain mitigation effect.

13. The biometric wearable system of claim 1 further comprising a transceiver,
wherein data comprising any one or more of: (1) the biometric signal data of the user, (2) the one or more user-specific biosignal patterns and/or a user-specific limb motion intent of the user, and/or (3) the learned user intent data, and/or (4) data associated with the biometric signal data of the user, is configured to be transmitted to and from a server remote to the biometric wearable device.

14. The biometric wearable system of claim 13, wherein the data provided to an artificial intelligence (AI) algorithm for training or retraining an AI model configured to analyze the biometric signal data to identify in real time or near real time the one or more user-specific biosignal patterns and/or a user-specific limb motion intent as detected when the user performs the intended limb motion.

15. A biometric wearable method for detecting intended limb motion and invoking pain mitigation, the biometric wearable method comprising:
sensing, by one or more biosensors, biometric signal data of a user while the user performs an intended limb motion of a given limb of the user, wherein each of the one or more biosensors form part of one or more sensor pods of a biometric wearable device worn by the user, and wherein each the one or more sensor pods includes one or more biofeedback indicators;
analyzing, by one or more processors, the biometric signal data to learn in real time or near real time a user-specific limb motion intent based on one or more user-specific biosignal patterns detected when the user performs the intended limb motion;
generating, by the one or more processors based on the user-specific limb motion intent, learned user intent data for at least one of: (1) visualizing a virtual limb on a display device, (2) visually controlling a graphical aspect on the display device, (3) visualizing the one or more user-specific biosignal patterns on the display device, and/or (4) visualizing the user-specific limb motion intent data on the display device; and
visualizing, by the one or more processors, the learned user intent data on the display device for invoking a pain mitigation effect with the user corresponding to the given limb.

16. The biometric wearable method of claim 15, wherein the pain mitigation effect reduces at least one ailment as experienced by the user comprising: (a) limb pain, (b) phantom limb pain, (c) complex regional pain syndrome (CRPS), (d) post-traumatic stress disorder (PTSD), or (e) burning sensation.

17. The biometric wearable method of claim 15, wherein the display device is a screen device or a headset device.

18. The biometric wearable method of claim 15 further comprising transmitting the learned user intent data via a transceiver to the display device.

19. The biometric wearable method of claim 15, wherein the virtual limb or the one or more user-specific biosignal patterns and/or specific limb motion intent data is visualized in a virtual environment.

20. The biometric wearable method of claim 15, wherein the display device is a display screen connected to the biometric wearable device, and wherein the virtual limb or one or more user-specific biosignal patterns and/or specific limb motion intent data is visualized on the display screen.

21. The biometric wearable method of claim 15 further comprising:
receiving an input from a computing device to configure or alter an operation or setting of the biometric wearable device; and/or
providing an indication of an operation or status of the biometric wearable device via at least one of the one or more biofeedback indicators of at least one of the one or more sensor pods.

22. The biometric wearable method of claim 15, wherein the one or more sensor pods is part of a wearable prosthetic liner interface and/or a partial sleeve interface.

23. The biometric wearable method of claim 15, wherein the one or more biofeedback indicators of each sensor pod are configured to provide an output to indicate amplitude information of a signal detected by the one or more biosensors of the sensor pods.

24. The biometric wearable method of claim 23, wherein the output of the one or more biofeedback indicators of each sensor pod comprises an illumination, in a predetermined pattern, to indicate amplitude information of a signal detected by the one or more biosensors of the sensor pods.

25. The biometric wearable method of claim 23, wherein the intended limb motion comprises a muscle bulge of the given limb of the user causing an increase of pressure of the given limb against the one or more sensor pods, wherein the increased pressure invokes a sensorimotor effect that enhances the pain mitigation effect.

26. The biometric wearable method of claim 25, wherein the increase in muscle bulge causes a corresponding increase in the user's biosignal output causing at least one of: (1) an illumination of a plurality of light emitting diodes of at least one sensor pod of the plurality of sensor pods, and/or (2) a frequency or vibration of actuators of at least one sensor pod of the plurality of sensor pods, wherein the increased pressure and the increase in the output invokes a sensorimotor effect that enhances the pain mitigation effect.

27. The biometric wearable method of claim 15 further comprising transmitting via a transceiver, to and from a server remote to the biometric wearable device, data comprising any one or more of: (1) the biometric signal data of the user, (2) the one or more user-specific biosignal patterns and/or a user-specific limb motion intent of the user, and/or (3) the learned user intent data, and/or (4) data associated with the biometric signal data of the user, is configured to be transmitted to and from a server remote to the biometric wearable device.

28. The biometric wearable method of claim 27 further comprising providing the server to an artificial intelligence (AI) algorithm for training or retraining an AI model configured to analyze the biometric signal data to identify in real time or near real time the one or more user-specific biosignal patterns and/or a user-specific limb motion intent as detected when the user performs the intended limb motion.

29. A tangible, non-transitory computer-readable medium storing instructions for detecting intended limb motion and invoking pain mitigation, that when executed by one or more processors cause the one or more processors to:
sense, by one or more biosensors, biometric signal data of a user while the user performs an intended limb motion of a given limb of the user, wherein each of the one or more biosensors form part of one or more sensor pods of a biometric wearable device worn by the user, and wherein each the one or more sensor pods includes one or more biofeedback indicators;
analyze, by one or more processors, the biometric signal data to learn in real time or near real time a user-specific limb motion intent based on one or more user-specific biosignal patterns detected when the user performs the intended limb motion;
generate, by the one or more processors based on the user-specific limb motion intent, learned user intent data for at least one of: (1) visualizing a virtual limb on a display device, (2) visually controlling a graphical aspect on the display device, (3) visualizing the one or more user-specific biosignal patterns on the display device, and/or (4) visualizing the user-specific limb motion intent data on the display device; and
visualize, by the one or more processors, the learned user intent data on the display device for invoking a pain mitigation effect with the user corresponding to the given limb.
